(19) United States
(12) Patent Application Publication
(10) Pub. No.: US 2025/0375497 A1
(43) Pub. Date: Dec. 11, 2025

(54) MIXED LINKAGE GLUCAN BIOSYNTHESIS BY CELLULOSE SYNTHASE-LIKE CSLF6 (1,3;1,4)-B-GLUCAN SYNTHASE

(71) Applicant: University of Virginia Patent Foundation, Charlottesville, VA (US)

(72) Inventors: Jochen Zimmer, Charlottesville, VA (US); Purushotham Naidu Pallinti, Charlottesville, VA (US)

(21) Appl. No.: 18/874,464

(22) PCT Filed: Jun. 23, 2023
(86) PCT No.: PCT/US2023/068965

§ 371 Date: Dec. 12, 2024

Related U.S. Application Data
(60) Provisional application No. 63/366,935, filed on Jun. 24, 2022.

(51) Int. Cl. A61K 36/899; C12N 9/10; C12N 15/82

(52) U.S. Cl. CPC A61K 36/899; C12N 9/1051; C12N 15/8246; C12Y 204/01

(57) ABSTRACT

Compositions, foodstuff and methods are provide herein from cereal plant or a part thereof, wherein the plant or part thereof has an altered (I,3;1,4)-p-glucan content as compared to a wild-type cereal plant or part thereof, wherein said, plant or part thereof carries one or more mutations in the CslF6 gene, wherein said mutated. CslF6 gene encodes a mutant CslF6 polypeptide, wherein said mutant CslF6 comprises at least one substitution, addition or deletion of an amino acid in a switch motif of CslF6, wherein the switch motif comprises SEQ ID NO: 14.

### PRIORITY APPLICATION

This application claims the benefit of priority to U.S. Provisional Patent Application Ser. No. 63/366,935, filed Jun. 24, 2022, the content of which is incorporated herein by reference in its entirety.

### GOVERNMENT GRANT SUPPORT

This invention was made with government support under DESC0001090 awarded by the U.S. Department of Energy. The government has certain rights in the invention.

### INCORPORATION BY REFERENCE OF SEQUENCE LISTING

A Sequence Listing is provided herewith as an xml file, “2345641.xml” created on Jun. 22, 2023 and having a size of 80,636 bytes. The content of the xml file is incorporated by reference herein in its entirety.

### BACKGROUND OF THE INVENTION

Cell walls are dynamic carbohydrate-rich extracellular matrices that define cell shape, mediate tissue organization and plant growth, and provide resilience to adverse environmental stresses. In the cell walls of terrestrial plants, cellulose forms a load-bearing network of microfibrils that are embedded in a complex composite of hemicelluloses, pectins, proteins and, in specific tissues, lignins (1).

### SUMMARY OF THE INVENTION

On embodiment provides a cereal plant (such as barley (Hordeum), wheat (Triticum), rice (Oryza), maize (Zea), rye (Secale), oat (Avena), sorghum (Sorghum), and Triticale, a rye-wheat hybrid) or a part thereof, wherein the plant or part thereof has an altered (1,3;1,4)-β-glucan content as compared to a wild-type cereal plant or part thereof, wherein said plant or part thereof carries one or more mutations in the CslF6 gene, wherein said mutated CslF6 gene encodes a mutant CslF6 polypeptide, wherein said mutant CslF6 comprises at least one substitution, addition or deletion of an amino acid in a switch motif of CslF6, wherein the switch motif comprises SEQ ID NO:14. In one embodiment, one or more amino acids are substituted in the switch motif. In another embodiment, the tyrosine of the switch motif is substituted with a histidine. In one embodiment, the substitution, addition or deletion results in a reduction of (1,3)-β-linkage formation as compared to wild type. In one embodiment, the altered plant (1,3;1,4)-β-glucan content results in a ratio DP3:DP4 of 1:2.5 to 1:32. In another embodiment, the ratio is 1.1:2 or 1.2:2 or 1.3:2 or 1.4:2 or 1.5:2 and so on. In one embodiment, the substitution, addition or deletion results in increase of (1,3)-β-linkage formation as compared to wild type.

One embodiment provides a composition comprising the plant or a part thereof described herein.

Another embodiment provides a food or drink product comprising the plant or a part thereof described herein or a composition described herein. In one embodiment, the food or drink product of claim 8, wherein the food or drink product is selected from a malt beverage, a flour, a syrup, a malt, a beer, a bagel, a biscuit, a bread, a bun, a croissant a dumpling, an English muffin, a muffin, a pita bread, a quick bread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut an egg roll, an extruded snack, a fruit and grain bar, a microwaveable snack product a nutritional bar, a pancake, a par-baked bakery product, a pretzel, a pudding, a granola-based product, a snack chip, a snack food, a snack mix, a waffle, a pizza crust, animal food or pet food.

One embodiment provides a food additive, bulking agent, dietary fiber, texturizing agent, preservative or probiotic agent made from the plant or part thereof as described herein.

One embodiment provides a method to treat or improve the health of an animal, comprising administering the plant or a part thereof as described herein or a composition as described herein or the food or drink product as described herein to the animal in need thereof so as to treat or improve said animal's health. In one embodiment, the metabolic health, bowel health or cardiovascular health is improved or the severity of metabolic, bowel or cardiovascular disease is decreased in said animal. In one embodiment, the animal is a mammal. In one embodiment, the mammal is a human.

### BRIEF DESCRIPTION OF THE DRAWINGS

FIG. 1A-1F. In vitro (1,3:1,4)-β-glucan biosynthesis by barley CslF6. (A) Size exclusion chromatography profile of GDN-solubilized CslF6. Inset: Coomassie stained SDS-PAGE of the indicated fractions. mAU: Absorbance at 280 nm (×10−3). (B) Catalytic activity of purified CslF6 measured upon incorporation of 3H-labeled glucose, as described previously (23). The product yield obtained in the presence of magnesium was set as 100%. (C) Time course of product accumulation. (D) Enzymatic degradation of the in vitro synthesized glucan. Material recovered without digestion was set as 100%. All error bars represent deviations from the means of at least three replicas. (E) HPLC size exclusion chromatography of in vitro synthesized and lichenase digested product (shown in green), (1,3)-β-glucanase (magenta), (1,4)-β-glucanase (black). The undigested sample is shown in blue and a standard cello-oligosaccharide mixture ranging from hexa- to monosaccharides is shown in red (labeled 6 to 1). The signals expressed as nano Refractive Index Units (nRIU). (F) Glycosyl linkage analysis of the CslF6-synthesized glucan. Shown is a gas chromatogram of the separated alditol acetates. The identity of the 3- and 4-linked as well as terminal glucosyl units was confirmed by electron-impact mass spectrometry (FIG. S2).

FIG. 2A-2D. Architecture of barley CslF6. (A) Representative 2D class averages of micelle solubilized CslF6. (B) Cartoon representation of CslF6. TM helices are colored blue to red from the N- to the C-terminus. The GT domain is shown in pale yellow, the PCR and CSR domains are colored brown and magenta, respectively. (C) Monomeric CslF6 forms a TM channel with a large lateral opening. The channel was rendered using a 2.5 Å radius probe calculated in HOLLOW (37) and displayed as an orange surface. (D) A poorly resolved glucan likely occupies the CslF6 channel. Bacterial CesA (PDB: 4P00) containing a nascent cellulose polymer (shown as cyan and red sticks) was overlayed with the CslF6 cryo-EM map (shown as semi-transparent gray surface).

FIG. 3A-3C. CslF6 adopts a CesA-like architecture. (A) CslF6 was superimposed with one protomer of the hybrid aspen CesA8 trimeric structure (PDB: 6WLB). CesA8 is shown as a semi-transparent cartoon colored dark violet. The other CesA8 subunits are shown as gray ribbons. CslF6's TM region is colored blue. (B) Conservation of the active site. The substrate-binding pocket is indicated by a dashed oval. (C) Sequence alignment of the switch motif from hybrid aspen CesA8 and barley CslF6 and CslD and localization in the 3-dimensional structure. Hybrid aspen CesA8 is superimposed and shown as in panel A. The nascent cellulose polymer present in PDB:6WLB is shown as cyan and red ball-and-sticks in all panels.

FIG. 4A-4F. CslF6 mutagenesis affecting (1,3:1,4)-β-glucan biosynthesis. (A) Polysaccharide products recovered from in vitro synthesis reactions with the CslF6 mutants, Y787H and CslF6-CesA8-sm (CslF6 with its switch motif replaced with the corresponding IF3 region from CesA8) in the presence of EDTA (E), no degrading enzymes (Nd), lichenase (Lic), and endo-(1,4)- and (1,3)-β-glucanase (1,4 and 1,3), respectively. Product yields are normalized to undigested samples. Error bars represent standard deviations from three independent replicas. Inset: Coomassie stained 7.5% SDS-PAGE of the purified mutants. (B and C) Permethylated alditol acetates obtained from linkage analyses of the products produced by the CslF6 Y787H (B) and swapped switch motif mutants (C). (D) Catalytic activity of the CslF6 D629G and I757L mutants as described for the wild type enzyme in FIG. 1. DPM: Disintegrations per minute. Inset: Coomassie stained 10% SDS-PAGE of the purified mutants. (E and F) Glycosyl linkage analyses of glucans produced by the indicated CslF6 point mutants. 2-Galp represents a contamination of unknown origin, also observed in control analyses of buffer or cellulose samples (FIG. S7B).

FIG. 5A-5E. Model of (1,3:1,4)-β-glucan biosynthesis. (A) The C6 hydroxyl groups of consecutive (1,3)- and (1,4)-β-linked glucosyl units point in approximately the same direction during translocation while consecutive (1,4)-β-linked glucosyl units are rotated by about 180 degrees. (B) CslF6's switch motif induces acceptor repositioning due to register-dependent interactions with the penultimate glucosyl unit, resulting in the formation of DP3 segments. The position of Tyr787 is shown at the point of the switch motif (black triangle). (C) Acceptor repositioning may also occur upon interactions with the third glucosyl unit, resulting in the formation of DP4 segments. (D) Acceptor repositioning due to interactions with the glucan's second and third glucosyl unit generates a polymer harboring DP3 and DP4 segments. (E) Model of a (1,3;1,4)-β-glucan as shown in panel D. (1,3)- and (1,4)-β-linked glucosyl units are shown in pale green and blue for their carbon atoms, respectively. The model was generated and refined using GLYCAM (www.glycam.org) and asterisks highlight C6 positions. Dashed lines indicate lichenase cleavage sites.

FIG. 6. Sequence alignment of CesAs, CslDs, and CslF6s from different plant species. IF3 harboring the switch motif is indicated by a gray cylinder. The switch motif is framed with a red box. Sequences were aligned using MUSCLE (44) and displayed in Jal View (45), colored dark to light blue for invariant and similar residues. SEQ ID NOs: 30-64.

### DETAILED DESCRIPTION

Mixed-linkage (1,3:1,4)-β-glucans are abundant polymers of cell walls of the Poaceae (i.e., grasses) and are generally absent in the walls of eudicots (2). The (1,3:1,4)-β-glucans of common cereal species consist of ˜25% and ˜75% of (1,3)- and (1,4)-β-linked glucosyl units, respectively, in an unbranched and unsubstituted polymer. The structure of the polysaccharide consists largely of cellotriosyl and cellotetraosyl units separated by single (1,3)-β-linkages (2). The (1,3)-linkages introduce flexible kinks at irregular intervals into the polysaccharide (2, 3). As a result, (1,3:1,4)-β-glucans do not easily align to form insoluble fibers, as is observed with the (1,4)-β-glucan produced by cellulose synthase (CesA). Due to their increased water solubility, (1,3:1,4)-β-glucans form highly viscous aqueous solutions. These properties provide important benefits in human health by reducing the risk of serious diseases, including colorectal cancer, type II diabetes, and coronary heart disease (4, 5), as well as food products.

The cell wall polysaccharides of cereal grains are an important dietary component in human nutrition, being a significant source of dietary fiber. Consumption of whole grain cereals, of which cell wall polysaccharides comprise about 10% by dry weight, is associated with a reduced risk of developing diseases such as type 2 diabetes, cardiovascular disease and colorectal cancer, as well as with other health benefits such as improved gastrointestinal health. Whole grains also have a relatively low glycemic index and are a rich source of other dietary components including vitamins, antioxidants and minerals, as well as starch as an energy source. The cell walls of grasses (Poaceae) including cereal grains are characterized by the presence of mixed linkage (1,3:1,4)-β-glucans.

The variability of (1,3:1,4)-β-glucan structures can be expressed in terms of their DP3:DP4 ratios, where DP is the degree of polymerization and DP3 and DP4 are easily measurable proportions of cellotriosyl and cellotetraosyl units, respectively (6). The DP3 and DP4 fragments are released upon degradation with lichenase, which is an endo-(1,3;1,4)-β-glucanase that hydrolyzes the (1,4)-β-linkage directly following, towards the reducing end of the polysaccharide, a (1,3)-β-linked glucosyl unit (7). Water-soluble (1,3;1,4)-β-glucans generally have DP3:DP4 ratios of about 2:1 to 3:1.

A major class of enzymes involved in the formation of plant cell wall polysaccharides are family-2 glycosyltransferases (GTs), including CesAs (8). The CesA enzyme couples cellulose synthesis with polysaccharide secretion through a transmembrane (TM) channel formed by its membrane-embedded region (16). The enzyme utilizes UDP-glucose as substrate and elongates and translocates the nascent cellulose polymer one glucosyl unit at a time (9). In the SN2-like substitution reaction (10), the C4 hydroxyl of cellulose's non-reducing end glucosyl unit (the acceptor) mediates a nucleophilic attack on the C1 carbon of the substrate's glucosyl residue (the donor). This reaction is facilitated by a base catalyst that deprotonates the acceptor during the nucleophilic attack (10). The base catalyst is formed by the aspartate residue of an invariant Thr-Glu-Asp motif of the active site that is in hydrogen bond distance to the acceptor's C4 hydroxyl (9).

The enzymes involved in (1,3:1,4)-β-glucan formation in grasses most likely utilize a similar reaction mechanism. They belong to the cellulose synthase-like CslF, CslH and CslJ groups of enzymes (11-13). Among those, the CslF6 enzyme, which is the predominant isoenzyme in developing barley grain, is closely related to CesAs, as reflected in a high degree of sequence similarity across the catalytic domains and TM regions, as well as similar predicted TM topologies (8, 14, 15). Specifically, the CslF clade is nested within the CslD subfamily and evolved following the CesA-CslD divergence (11, 16) Although the functions of all CslD enzymes have not been defined unequivocally, some have been implicated in cellulose formation in tip-growing cells (13, 17, 18).

The similarity of (1,3:1,4)-β-glucan synthases with CesA and the unusual and variable structural features of (1,3:1,4)-β-glucans raise two important, unanswered questions about their biosynthesis. First, can both linkage types be inserted into the polysaccharide by a single enzyme, or are multiple enzymes required (19-21)? Second, how does the enzyme or enzyme complex non-randomly incorporate single (1,3)-β-linkages into a (1,4)-β-backbone, while the cellotriosyl and cellotetraosyl units between these (1,3)-linkages are arranged at random?

To shed light on these fundamental questions in cell wall polysaccharide biosynthesis, barley (Hordeum vulgare) CslF6 was expressed in Sf9 insect cells to test its ability to synthesize a (1,3:1,4)-β-glucan in the absence of other plant-derived components. The results unambiguously demonstrate the formation of (1,3;1,4)-β-glucans with DPs of 25 to 100, and with structures similar to (1,3;1,4)-β-glucans isolated from barley grain. Additionally, a medium resolution cryogenic electron microscopy (cryo-EM) structure of the monomeric enzyme was determined at an intermediate state during polymer biosynthesis. The structure and primary sequence analyses identified a switch motif at the entrance to the enzyme's TM channel that is needed for (1,3)-β-linkage formation.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

A “cereal” plant, as defined herein, is a member of the Poaceae plant family, cultivated primarily for their starch-containing seeds or kernels. Cereal plants include, but are not limited to barley (Hordeum), wheat (Triticum), rice (Oryza), maize (Zea), rye (Secale), oat (Avena), sorghum (Sorghum), and Triticale, a rye-wheat hybrid.

As used herein, the term (1,3;1,4)-β-glucan”, also referred to as “β-glucan” refers to an essentially linear polymer of unsubstituted and essentially unbranched β-glucopyranosyl monomers covalently linked mostly through (1,4)-linkages with some (1,3)-linkages. The glucopyranosyl residues, joined by (1-3)- and (1-4)-linkages, are arranged in a non-repeating but nonrandom fashion—i.e., the (1,4)- and (1,3)-linkages are not arranged randomly, but equally they are not arranged in regular, repeating sequences. Most (about 90%) of the (1-3)-linked residues follow 2 or 3 (1-4)-linked residues in oat and barley β-glucan. Typically, the β-glucan polymers have at least 1000 glycosyl residues and adopt an extended conformation in aqueous media. The ratio of tri- to tetra-saccharide units (DP3/DP4 ratio) varies among species and therefore is characteristic of β-glucan from a species.

The term “DP” as used herein refers to the degree of polymerization, and indicates the number of α-1,4-linked glucose units in amylopectin side chains. Thus, by way of example DP3 refers to amylopectin side chains consisting of a sequence of 3 α-1,4-linked glucose units. Similarly, DP4 refers to amylopectin side chains consisting of a sequence of 4 α-1,4-linked glucose units. The term “DP3:DP4 ratio” of (1,3;1,4)-β-glucans as used herein refers to the ratio of amylopectin side chains consisting of a sequence of 3 α-1,4-linked glucose units and of amylopectin side chains consisting of a sequence of 4 α-1,4-linked glucose units within said (1,3;1,4)-β-glucans. The DP3:DP4 ratio may be determined by digesting (1,3;1,4)-β-glucans with lichenase followed by quantification of released DP3 and DP4 oligomers e.g., by HPAEC-PAD.

“(1,3;1,4)-β-glucan content” as used herein may be determined by any useful method. The “(1,3;1,4)-β-glucan content” is determined as the sum of the content of Glc-β-(1→4)-Glc-β-(1→3)-Glc (DP3) and Glc-β-(1→4)-Glc-β-(1→4)-Glc-β-(1→3)-Glc (DP4) oligomers. The content of DP3 and DP4 oligomers may e.g., be determined by lichenase digestion of (1,3;1,4)-β-glucans followed by quantification e.g., by High-performance anion exchange chromatography with pulsed amperometric detection (HPAEC-PAD).

As used herein, the term “by weight” or “on a weight basis” refers to the weight of a substance, for example, β-glucan, as a percentage of the weight of the material or item comprising the substance. This is abbreviated herein as “w/w”.

The term “plant(s)” and “plant part(s), as used herein refers to whole plants, or any substance which is present in, obtained from, derived from, or related to a plant or a plant part, such as for example, plant organs (e.g. leaves, stems, roots, flowers), single cells (e.g. pollen), seeds or grain, plant cells including for example tissue cultured cells, products produced from the plant such as flour, grain, starch and the like. Plantlets and germinated seeds from which roots and shoots have emerged are also included within the meaning of “plant”. The term “plant parts” as used herein refers to one or more plant tissues or organs which are obtained from a whole plant. Plant parts include vegetative structures (for example, leaves, stems), roots, floral organs/structures, seed (including embryo, endosperm, and seed coat), plant tissue (for example, vascular tissue, ground tissue, and the like), cells and progeny of the same. The term “plant cell” as used herein refers to a cell obtained from a plant or in a plant, and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, pollen, and various forms of aggregations of plait cells in culture, such as calli. Plant tissues in or from seeds such as wheat grain are seed coat, endosperm, scutellum, aleurone layer and embryo.

Procedures such as crossing plants, self-fertilizing plants or marker-assisted selection are standard procedures and well known in the art. By the term “progeny” as used herein is meant a plant, which directly or indirectly is offspring of a given plant. Thus, progeny is not confined to direct off-spring but also includes off-spring after numerous generations. In general, progeny of a plant carrying a specific mutation also carries that specific mutation. Thus, progeny of a plant carrying a specific mutation in the CslF6 gene also carry that specific mutation.

The term “transgenic plant” as used herein refer to a plant that contains a gene construct (“transgene”) not found in a wild-type plant of the same species, variety or cultivar. That is, transgenic plants (transformed plants) contain genetic material that they did not contain prior to the transformation. A “transgene” as referred to herein has the normal meaning in the art of biotechnology and refers to a genetic sequence which has been produced or altered by recombinant DNA or SNA technology and which has been introduced into the plant cell. The transgene may include genetic sequences obtained from or derived from a plant cell or a non-plant source, or a synthetic sequence. Typically, the transgene has been introduced into the wheat plant by human manipulation such as, for example, by transformation but any method can be used, as one of skill in the art recognizes. The genetic material is typically stably integrated into the genome of the plant. The introduced genetic material may comprise sequences that naturally occur in the same species but in a rearranged order or in a different arrangement of elements, for example an antisense sequence. Plants containing such sequences are included herein in “transgenic plants”. Transgenic plants as defined herein include all progeny of an initial transformed and regenerated plant which has been genetically modified using recombinant techniques, where the progeny comprise the transgene. Such progeny may be obtained by self-fertilization of the primary transgenic plant or by crossing such plants with another plant of the same species. In an embodiment, the transgenic plants are homozygous for each and every gene that has been introduced (transgene) so that their progeny do not segregate for the desired phenotype. In some embodiments, the transgenes(s) in the transgenic plant are present at only a single genetic locus so that they are inherited together in all progeny. Transgenic plant parts include all parts and cells of said plants which comprise the transgene(s) such as, for example, grain, cultured tissues, callus and protoplasts. A “non-transgenic plant” is one which has not been genetically modified by the introduction of genetic material by recombinant DNA techniques.

Point mutations can also be generated in the cereal plants by methods available to an art worker. Amino acid sequence mutants of the polypeptides of the present invention can be prepared by introducing appropriate nucleotide changes into a nucleic acid of the present invention or by mutagenesis in vivo such as by chemical or radiation treatment. Such mutants include, for example, deletions, insertions or substitutions of residues within the amino acid sequence. The polynucleotides of the invention may be subjected to DNA shuffling techniques as described or other in vitro methods to produce altered polynucleotides which encode polypeptide variants.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place.

The present invention makes use of vectors for production, manipulation or transfer of chimeric genes or genetic constructs. By “vector” is meant a nucleic acid molecule, preferably a DNA molecule derived, for example, from a plasmid, bacteriophage or plant virus, into which a nucleic acid sequence may be inserted. A vector preferably contains one or more unique restriction sites and may be capable of autonomous replication in a defined host cell including a target cell or tissue or a progenitor cell or tissue thereof or be integrate into the genome of the defined host such that the cloned sequence is reproducible. Accordingly, the vector may he an autonomously replicating vector, he., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene that can be used for selection of suitable transformants, or sequences that enhance transformation of prokaryotic or eukaryotic cells such as T-DNA or P-DNA sequences. Examples of such resistance genes and sequences are well known to those of skill in the art.

A number of techniques are available for the introduction of nucleic acid molecules to plant, well known to workers to in the art. The term “transformation” as used hereto means alteration of the genotype of a cell, for example a bacterium or a plant, by the introduction of a foreign or exogenous nucleic acid. By “transformant” is meant an organism so altered, introduction of DNA into a plant by crossing parental plants or by mutagenesis per se is not included to transformation. As used herein the term “transgenic” refers to a genetically modified plant to which the endogenous genome is supplemented or modified by the random or site-directed integration, or stable maintenance in a replicable non-integrated form, of an introduced foreign or exogenous gene or sequence. By “transgene” is meant a foreign or exogenous gene or sequence that is introduced into a plant. The nucleic acid molecule may be replicated as an extrachromosomal element or is preferably stably integrated into the genome of the plant. By “genome” is meant the total inherited genetic complement of the cell, plant or plant part, and includes chromosomal DNA, plastid DNA, mitochondrial DNA and extra chromosomal DNA molecules. Two methods are commonly used to deliver the DNA: T-DNA transfer using Agrobacterium tumefaciens or related bacteria and direct introduction of DNA via particle bombardment, although other methods have been used to integrate DNA sequences into cereals. Another method is high velocity ballistic penetration by small molecules. It will be apparent to the skilled person that the particular choice of a transformation system to introduce a nucleic acid construct into plant cells is not essential to or a limitation of the invention, provided it achieves an acceptable level of nucleic acid transfer. Such techniques are well known in the art.

The term “amino acid” as used herein refers to a proteinogenic amino acid. Preferably, the proteinogenic amino acids is one of the 20 amino acids encoded by the standard genetic code. The IUPAC one and three letter codes are used to name amino acids.

The term “charged amino acid” as used herein refers to amino acids with electrically charged side chains. Preferably, the charged amino acid is selected from the group consisting of Arg, His, Lys, Asp and Glu. Negatively charged amino acids are preferably selected from the group consisting of Asp and Glu.

The term “non-polar amino acid” as used herein refers to amino acids with a hydrophobic side chains. The non-polar amino acid is selected from the group consisting of Ala, Val, Ile, Leu, Met, Phe, Tyr, Trp and Gly.

The term “polar amino acid” as used herein refers to amino acids with polar, uncharged side chains. Preferably, the polar amino acid is selected from the group consisting of Ser, Thr, Asn and Gln.

The term “amino acid corresponding to X” is used herein to describe amino acids of a given polypeptide (e.g., a mutant CslF6 polypeptide) in relation to amino acids of a reference polypeptide (e.g., CslF6 of SEQ ID NO:15). Following alignment between said polypeptide and the reference polypeptide, an amino acid is corresponding to X if it is in the same position as X in said alignment.

The term “sequence identity” as used herein refers to the % of identical amino acids or nucleotides between a candidate sequence and a reference sequence following alignment. Thus, a candidate sequence sharing 80% amino acid identity with a reference sequence requires that, following alignment, 80% of the amino acids in the candidate sequence are identical to the corresponding amino acids in the reference sequence. Identity according to the present invention is determined by aid of computer analysis, such as, without limitations, the Clustal Omega computer alignment program for alignment of polypeptide sequences (Sievers et al. (2011 Oct. 11) Molecular Systems Biology 7:539, PMID: 21988835; Li et al. (2015 Apr. 6) Nucleic Acids Research 43 (W1):W580-4 PMID: 25845596; McWilliam et al., (2013 May 13) Nucleic Acids Research 41 (Web Server issue):W597-600 PMID: 23671338, and the default parameters suggested therein. The Clustal Omega software is available from EMBL-EBI at https://www.ebi.ac.uk/Tools/msa/clustalo/. Using this program with its default settings, the mature (bioactive) part of a query and a reference polypeptide are aligned. The number of fully conserved residues are counted and divided by the length of the reference polypeptide. The MUSCLE or MAFFT algorithms may be used for alignment of nucleotide sequences. Sequence identities may be calculated in a similar way as indicated for amino acid sequences. Sequence identity as provided herein is thus calculated over the entire length of the reference sequence. The sequences disclosed herein can have at least 75%, 80%, 85%, 90%, 95%, 96%, 96%, 98%, or 100% sequence identity as compared to the reference sequence.

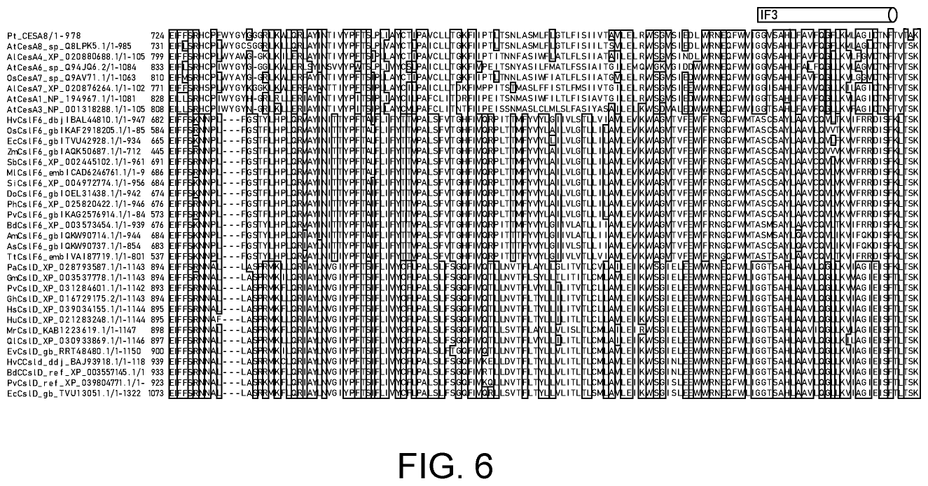

The term “wild type CslF6” as used herein refers to a gene encoding a polypeptide of SEQ ID NO:15 (barley) or the others as disclosed in FIG. 6 (full length amino acid sequences provided as SEQ ID NOs: 15 and 17-29).

By “encoding” or “encoded”, in the context of a specified nucleic acid, is meant comprising the information for translation into the specified protein. A nucleic acid or polynucleotide encoding a protein may comprise non-translated sequences, e.g., introns, within translated regions of the nucleic acid, or may lack such intervening non-translated sequences, e.g., in cDNA. The information by which a protein is encoded is specified by the use of codons.

As used herein, “expression” in the context of nucleic acids is to be understood as the transcription and accumulation of mRNA. “Expression” used in the context of proteins refers to translation of mRNA into a polypeptide.

The terms “polypeptide” and “protein” are generally used interchangeably herein. The terms “proteins” and “polypeptides” as used hereto also include variants, mutants, modifications and/or derivatives of tire polypeptides of the invention as described herein.

The term “gene” means the segment of DNA involved in producing a polypeptide chain; it includes regions preceding and following the coding region (promoter and terminator). Furthermore, plant genes generally consist of exons interrupted by introns.

“Mutations” include deletions, insertions, substitutions, transversions, and point mutations in the coding and noncoding regions of a gene. Deletions may be of the entire gene, or of only a portion of the gene. Point mutations may concern changes of one base pair, and may for result in premature stop codons, frameshift mutations, mutation of a splice site or amino acid substitutions. A gene comprising a mutation may be referred to as a “mutant gene”. If said mutant gene encodes a polypeptide with a sequence different to the wild type, said polypeptide may be referred to as a “mutant polypeptide”.

By the term “plant product” is meant a product resulting from the processing of a plant or plant material. Said plant product may thus, for example, be a fermented or non-fermented beverage, a food, or a feed product.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

### CSlF6

The present invention provides a plant or a part thereof, wherein said barley plant or part thereof has altered (1,3;1,4)-β-glucan content as compared to a wildtype plant, wherein said plant carries a mutation in the CslF6 gene and wherein said mutated CslF6 gene encodes a mutant CslF6 polypeptide.

The sequence of a wild type cellulose synthase-like CslF6 (CslF6) coding sequence from barley Hordeum vulgare subsp. vulgare is provided herein as SEQ ID NO:15.

```
      1 MAPAVAGGGR VRSNEPVAAA AAAPAASGKP CVCGFQVCAC TGSAAVASAA SSLDMDIVAM
    61 GQIGAVNDES WVGVELGEDG ETDESGAAVD DRPVFRTEKI KGVLLHPYRV LIFVRLIAFT
  121 LFVIWRISHK NPDAMWLWVT SICGEFWFGF SWLLDQLPKL NPINRVPDLA VLRQRFDRPD
  181 GTSTLPGLDI FVTTADPIKE PILSTANSVL SILAADYPVD RNTCYVSDDS GMLLTYEALA
  241 ESSKFATLWV PFCRKHGIEP RGPESYFELK SHPYMGRAQD EFVNDRRRVR KEYDEFKARI
  301 NSLEHDIKQR NDGYNAAIAH SQGVPRPTWM ADGTQWEGTW VDASENHRRG DHAGIVLVLL
  361 NHPSHRRQTG PPASADNPLD LSGVDVRLPM LVYVSREKRP GHDHQKKAGA MNALTRASAL
  421 LSNSPFILNL DCDHYINNSQ ALRAGICFMV GRDSDTVAFV QFPQRFEGVD PTDLYANHNR
  481 IFFDGTLRAL DGMQGPIYVG TGCLFRRITV YGFDPPRINV GGPCFPRLAG LFAKTKYEKP
  541 GLEMTTAKAK AAPVPAKGKH GFLPLPKKTY GKSDAFVDTI PRASHPSPYT AAAEGIVADE
  601 ATIVEAVNVT AAAFEKKTGW GKEIGWVYDT VTEDVVTGYR MHIKGWRSRY CSIYPHAFIG
  661 TAPINLTERL FQVLRWSTGS LEIFFSKNNP LFGSTYLHPL QRVAYINITT YPFTAIFLIF
  721 YTTVPALSFV TGHFIVQRPT TMFYVYLGIV LSTLLVIAVL EVKWAGVTVF EWFRNGQFWM
  781 TASCSAYLAA VCQVLTKVIF RRDISFKLTS KLPSGDEKKD PYADLYVVRW TPLMITPIII
  841 IFVNIIGSAV AFAKVLDGEW THWLKVAGGV FFNFWVLFHL YPFAKGILGK HGKTPVVVLV
  901 WWAFTFVITA VLYINIPHMH TSGGKHTTVH GHHGKKLVDT GLYGWLH
SEQ ID NO: 15 is code by SEQ ID NO: 16
      1 atggcgccag cggtggccgg agggggccgc gtgcggagca atgagccggt tgctgctgct
    61 gccgccgcgc cggcggccag cggcaagccc tgcgtgtgcg gcttccaggt ttgcgcctgc
  121 acggggtcgg ccgcggtggc ctccgccgcc tcgtcgctgg acatggacat cgtagccatg
  181 gggcagatcg gcgccgtcaa cgacgagagc tgggtgggcg tggagctcgg cgaagatggc
  241 gagaccgacg aaagcggtgc cgccgttgac gaccgccccg tattccgcac cgagaagatc
  301 aagggtgtcc tcctccaccc ctaccggtac gtcctgctcc cacaactaaa cagaaactcc
  361 ctatatctgc gtcacactca acaattaatc caactaagtc tctctactac tatagtattt
  421 atttttactc tctatctgca caacaagcgc tactacaatt aacccaacaa gcaccacgcc
  481 aggttgacag tcaggataat ttgatcttga ccggagtaag tactagtact aggtcggtgt
  541 taatcagagt aattattgca ctagttaatt aaaatttgag taatccgaga caggtgcacg
  601 ttagggccgg gccaatgatc gctcgaatcc acccaaaata gcgcgtcccg gtgtgggctg
  661 tcggctcggt gcttcttcct tccattttac tagtcgcagt cactgcagct tgggcccacg
  721 ggaggggacg ttagccgttg ggcctgccag gcatgtgggc cccggtggcc accctggcgg
  781 ctcaggtggg ccccggtggc caccctggcg gctcataaat ccttgctact ttggagctgt
  841 aatggacgct ctgcaatagc aataggaatc cgaggtgaaa cgacgacagt gggcatggca
  901 tggcttgcat gtgaatccaa gccacatcat taaaagcatc ctccctgggc acgtcgcggt
  961 gagaaagttg gataaacttt tgggggttcg gacaagatga gaaaaagcaa gtaacatggc
1021 ccttttttgg caccgaagaa atcctatgta cggcgagttt tttctgcatc tagttatggg
1081 tagatgtacg ttagtttttg ttgagcgttt tatgtgctac atatatggag aaaaagagaa
1141 aaatattatc atgtcatgtc atgccatgag aagaaaaaga agtgttgcca ccgctcgaat
1201 gcctcttttt tctttcggaa ggatgcgtga gtcatgttgg caccgagaaa agccatatta
1261 aagtggcaga gttacaactc agaataaatg cgggtgttac aaaaacacta ggatatgtga
1321 agggcactcg gcacaacact ttaagactac acaattgaaa aagtgtactc tctgtatcta
1381 aataattata attgaaaaga attaatctat atatgaaagt agtaatgatt agcggtagaa
1441 ggttccaacg acttttttgg cgccaatagc aagaagaaag aaaaagaaaa aaatctttac
1501 tgtacagtaa cgagaaaaga ggcccattaa tagagcaacg aatccagcgg caccacctct
1561 ggcggtcacg tccatgccct cgcacgacgg atgaggcccg gggggtccta ctgacagccg
1621 aagcatgtcg gtgctcaaac acggcgccgt ttgctgccaa gtgtgccagc tcgcactcat
1681 tgacttgcca gctctctcct tggttgtcaa tgagaacatg atgccttttg gcatttgcaa
1741 acttattaaa actagctgtc gtccgatagg gaaaagaaaa gaaaagaaaa gaataagaaa
1801 aaaaggacaa agagaaaaga tgaacatggc gcatgttccc tccaataatt gcaggcacca
1861 acactgggtc gattaatcca acaacaatat tttactatac cagacgagag tacagtagtc
1921 gggtgatgat ggactgtaac tgactgagta tgaatgactg taatgcaggg tgctgatttt
1981 cgttcgtctg atcgccttca cgctgttcgt gatctggcgt atctcccaca agaacccaga
2041 cgcgatgtgg ctgtgggtga catccatctg cggcgagttc tggttcggtt tctcgtggct
2101 gctggatcag ctgcccaagc tgaaccccat caaccgcgtg ccggacctgg cggtgctgcg
2161 gcagcgcttc gaccgccccg acggcacctc cacgctcccg gggctggaca tcttcgtcac
2221 cacggccgac cccatcaagg agcccatcct ctccaccgcc aactcggtgc tctccatcct
2281 ggccgccgac taccccgtgg accgcaacac atgctacgtc tccgacgaca gtggcatgct
2341 gctcacctac gaggccctgg cagagtcctc caagttcgcc acgctctggg tgcccttctg
2401 ccgcaagcac gggatcgagc ccaggggtcc ggagagctac ttcgagctca agtcacaccc
2461 ttacatgggg agagcccagg acgagttcgt caacgaccgc cgccgcgttc gcaaggagta
2521 cgacgagttc aaggccagga tcaacagcct ggagcatgac atcaagcagc gcaacgacgg
2581 gtacaacgcc gccattgccc acagccaagg cgtgccccgg cccacctgga tggcggacgg
2641 cacccagtgg gagggcacat gggtcgacgc ctccgagaac caccgcaggg gcgaccacgc
2701 cggcatcgta ctggtcagta tccatccatc tttctgctgc ttatattact cttaggttac
2761 tcttatcgtc tctttcctat accgtacatg catgcatgct gctattcttg gaatcgtggt
2821 tggttactac tccaccatgc aaaaataaca agaagaggaa tcttggttag ttagggcctc
2881 gttgttatat tagtggccat ctgatgtgat gcctgccggc tgtgcccatc catatccatg
2941 gaagatttcg acagaatcga cgtggtgata gttgagagtg caaccaccac ccagagccag
3001 ccaagcacat gcatgcttct cttctcgtct cgtcgtgtgg ccagcagcgc attcatgcta
3061 ttgctgtgac gagggaggaa tggtggttgg ggtggtcctt tccccccgac agcactacag
3121 cctccacttt atgacccatt taattcatcg gccctgcttt gttgtaaccg ccttctcacc
3181 tcaatcaatc attcattcat tcataagttt actcactctt tgttactact cgaaccacta
3241 atcaggaagg agtaggagta atgcagattt actattaaca gttaaaggag taaaaagaag
3301 gaagcacaat tacagaacct tgtttttttt tactactgta cgtaaggtgt aagaatggag
3361 tgctgacaga gaatggatgc aggtgctgct gaaccacccg agccaccgcc ggcagacggg
3421 cccgccggcg agcgctgaca acccactgga cttgagcggc gtggatgtgc gtctccccat
3481 gctggtgtac gtgtcccgtg agaagcgccc cgggcacgac caccagaaga aggccggtgc
3541 catgaacgcg cttacccgcg cctcggcgct gctctccaac tcccccttca tcctcaacct
3601 cgactgcgat cattacatca acaactccca ggcccttcgc gccggcatct gcttcatggt
3661 gggacgggac agcgacacgg ttgccttcgt ccagttcccg cagcgcttcg agggcgtcga
3721 ccccaccgac ctctacgcca accacaaccg catcttcttc gacggcaccc tccgtgccct
3781 ggacggcatg cagggcccca tctacgtcgg cactgggtgt ctcttccgcc gcatcaccgt
3841 ctacggcttc gacccgccga ggatcaacgt cggcggtccc tgcttcccca ggctcgccgg
3901 gctcttcgcc aagaccaagt acgagaagcc cgggctcgag atgaccacgg ccaaggccaa
3961 ggccgcgccc gtgcccgcca agggtaagca cggcttcttg ccactgccca agaagacgta
4021 cggcaagtcg gacgccttcg tggacaccat cccgcgcgcg tcgcacccgt cgccctacac
4081 cgcggcggct gaggggatcg tggccgacga ggcgaccatc gtcgaggcgg tgaacgtgac
4141 ggccgccgcg ttcgagaaga agaccggctg gggcaaagag atcggctggg tgtacgacac
4201 cgtcacggag gacgtggtca ccggctaccg gatgcatatc aaggggtggc ggtcacgcta
4261 ctgctccatc tacccacacg ccttcatcgg caccgccccc atcaacctca cggagaggct
4321 cttccaggtg ctccgctggt ccacgggatc cctcgagatc ttcttctcca agaacaaccc
4381 gctcttcggc agcacatacc tccacccgct gcagcgcgtc gcctacatca acatcaccac
4441 ttaccccttc accgccatct tcctcatctt ctacaccacc gtgccggcgc tatccttcgt
4501 caccggccac ttcatcgtgc agcgcccgac caccatgttc tacgtctacc tgggcatcgt
4561 gctatccacg ctgctcgtca tcgccgtgct ggaggtcaag tgggccgggg tcacagtctt
4621 cgagtggttc aggaacggcc agttctggat gacagcaagt tgctccgcct acctcgccgc
4681 cgtctgccag gtgctgacca aggtgatatt ccggcgggac atctccttca agctcacatc
4741 caagctaccc tcgggagacg agaagaagga cccctacgcc gacctctacg tggtgcgctg
4801 gacgccgctc atgatcacac ccatcatcat catcttcgtc aacatcatcg gatccgccgt
4861 ggccttcgcc aaggttctcg acggcgagtg gacgcactgg ctcaaggtcg ccggcggcgt
4921 cttcttcaac ttctgggtgc tcttccacct ctaccccttc gccaagggca tcctggggaa
4981 gcacggaaag acgccagtcg tggtgctcgt ctggtgggca ttcaccttcg tcatcaccgc
5041 cgtgctctac atcaacatcc cccacatgca tacctcggga ggcaagcaca caacggtgca
```

The sequence of a wild type cellulose synthase-like CslF6 (CslF6) coding sequence from Oryza sativa is provided herein as SEQ ID NO: 17.

```
    1 MVIGCCRVLI FVRLIAFTLF VIWRIEHKNP DAMWLWVTSI AGEFWFGFSW LLDQLPKLNP
  61 INRVPDLAVL RRRFDHADGT SSLPGLDIFV TTADPIKEPI LSTANSILSI LAADYPVDRN
121 TCYLSDDSGM LLTYEAMAEA AKFATLWVPF CRKHAIEPRG PESYFELKSH PYMGRAQEEF
181 VNDRRRVRKE YDDFKARING LEHDIKQRSD SYNAAAGVKD GEPRATWMAD GSQWEGTWIE
241 QSENHRKGDH AGIVLVLLNH PSHARQLGPP ASADNPLDFS GVDVRLPMLV YVAREKRPGC
301 NHQKKAGAMN ALTRASAVLS NSPFILNLDC DHYINNSQAL RAGICFMLGR DSDTVAFVQF
361 PQRFEGVDPT DLYANHNRIF FDGTLRALDG LQGPIYVGTG CLFRRITLYG FEPPRINVGG
421 PCFPRLGGMF AKNRYQKPGF EMTKPGAKPV APPPAATVAK GKHGFLPMPK KAYGKSDAFA
481 DTIPRASHPS PYAAEAAVAA DEAAIAEAVM VTAAAYEKKT GWGSDIGWVY GTVTEDVVTG
541 YRMHIKGWRS RYCSIYPHAF IGTAPINLTE RLFQVLRWST GSLEIFFSRN NPLFGSTFLH
601 PLQRVAYINI TTYPFTALFL IFYTTVPALS FVTGHFIVQR PTTMFYVYLA IVLGTLLILA
661 VLEVKWAGVT VFEWFRNGQF WMTASCSAYL AAVLQVVTKV VFRRDISFKL TSKLPAGDEK
721 KDPYADLYVV RWTWLMITPI IIILVNIIGS AVAFAKVLDG EWTHWLKVAG GVFFNFWVLF
781 HLYPFAKGIL GKHGKTPVVV LVWWAFTFVI TAVLYINIPH IHGPGRHGAA SPSHGHHSAH
841 GTKKYDFTYA WP
```

The sequence of a wild type cellulose synthase-like CslF6 (CslF6) coding sequence from Eragrostis curvula provided herein as SEQ ID NO: 18.

```
    1 MAPGVGDGRR NNGEVVPAGG GRCVCGFQVC ACAGAAAVAS AASSADMDKM ALAATATEGQ
  61 IGAVNDESWV AVDLSDDLSG DGADAGVALE DRPVFRTEKI KGILLHPYRV LIFVRLIAFS
121 LFVVWRISHK NPDAISGSVS PGCWINSKTE PDQPRPGPGG APAALRPGRR HIPPAGLDIF
181 VTTADPFKEP ILSTANSILS ILAADYPVEK NTCYLSDDSG MLLTYEAMAE AAKFATVWVP
241 FCRKHGIEPR GPESYFELKS HPYMGRAQED FVNDRRRVRK EYDEFKARIN GLEHDIKQRS
301 DGYNANVKDG EPRATWMSDG TQWQGTWVEP SENHRKGDHA GIVLVLLNHP SHTRQLGPPA
361 SADNPLDFSL VDVRLPMLVY VAREKRPGHN HQKKAGAMNA LTRASAVLSN SPFILNLDCD
421 HYINNSQALR AGICFMLGRD SDTVAFVQFP QRFEGVDPTD LYANHNRIFF DGTLRALDGM
481 QGPIYVGTGC LFRRTTLYGF DPPRINVGGP CFPMLGGMFA KTKYEKPGLE LTTKAATIAK
541 GKHGFLPMPK KSYGKSDAFV DTIPKASHPS PYSAAEAAVV ADEAAIAEAV AVCTAAYEKK
601 TGWGSSIGWV YGTVTEDVVT GYRMHIKGWR SRYCSIYPHA FIGTAPINLT ERLYQVLRWS
661 TGSLEIFFSK NNPLFGSTFL HPLQRVAYIN ITTYPFTAIF LIFYTTVPAL SFVTGHFIVQ
721 RPTTMFYVYL LIVLGTLLIL AVLEVKWAGV TVFEWFRNGQ FWMTASCSAY LAAVLQVLFK
781 VVFRRDISFK LTSKQPAGDE KKDPYADLYV VRWTWLMVTP IIIILVNIIG SAVAFAKVLD
841 GEWTHWLKVA GGVFFNFWVL FHLYPFAKGL LGKHGKTPVV VLVWWAFTFV ITAVLYINIP
901 HFHASGGGHH AAKHGGHGAH HGTKHLDHVF FGWP
```

The sequence of a wild type cellulose synthase-like CslF6 (CslF6) coding sequence from Zea mays provided herein as SEQ ID NO: 19.

```
    1 MLLTYEAMAE AAKFATVWVP FCRKHGIEPR GPESYFELKS HPYMGRSQED FVNDRRRVRR
  61 DYDEFKARIN GLENDIRQRS DAYNAARGLK DGEPRATWMA DGTQWEGTWV EPSENHRKGD
121 HAGIVLVLLN HPSHSRQLGP PASADNPLDL SMVDVRLPML VYVSREKRPG HNHQKKAGAM
181 NALTRCSAVL SNSPFILNLD CDHYINNSQA LRAGICFMLG RDSDTVAFVQ FPQRFEGVDP
241 TDLYANHNRI FFDGTLRALD GMQGPIYVGT GCLFRRITLY GFDPPRINVG GPCFPSLGGM
301 FAKTKYEKPG LELTTKAAVA KGKHGFLPMP KKSYGKSDAF ADTIPMASHP SPFAAAAAVV
361 AEEATIAEAV AVCAAAYEKK TGWGSDIGWV YGTVTEDVVT GYRMHIKGWR SRYCSIYPHA
421 FIGTAPINLT ERLFQVLRWS TGSLEIFFSR NNPLFGSTFL HPLQRVAYIN ITTYPFTAIF
481 LIFYTTVPAL SFVTGHFIVQ RPTTMFYVYL AIVLGTLLIL AVLEVKWAGV TVFEWFRNGQ
541 FWMTASCSAY LAAVCQVLVK VVFRRDISFK LTSKQPAGDE KKDPYADLYV VRWTWLMVTP
601 IIIILVNIIG SAVAFAKVLD GEWTHWLKVA GGVFFNFWVL FHLYPFAKGI LGRHGKTPVV
661 VLVWWAFTFV ITAVLYINIP HIHGPGGKHG GAIGKHGAAH HGKKFDGYYL WP
```

The sequence of a wild type cellulose synthase-like CslF6 (CslF6) coding sequence from Sorghum bicolor provided herein as SEQ ID NO: 20.

```
    1 MAPGGGDGRR NGEGQQQANG NNNNNNSNAK AKHGCVCGFP VCACAGAAAV ASAASSADMD
  61 RVAAAQTEGQ IGAVNDESWI AVDLSDDLSG DGGGADPGVA IEDRPVFRTE KIKGILLHPY
121 RVLIFVRLIA FTLFVIWRIS HRNPDAMWLW VTSIAGEFWF GFSWLLDQLP KLNPINRVPD
181 LAVLRQRFDR ADGTSRLPGL DIFVITADPF KEPILSTANS ILSILAADYP VERNTCYLSD
241 DSGMLLTYEA MAEAAKFATV WVPFCRKHGI EPRGPESYFE LKSHPYMGRS QEDFVNDRRR
301 VRKEYDEFKA RINGLEHDIK QRSDAFNAAR GLKDGEPRAT WMADGNQWEG TWVEPSENHR
361 KGDHAGIVYV LLNHPSHSRQ LGPPASADNP LDFSMVDVRL PMLVYVSREK RPGFNHEKKA
421 GAMNALTRCS AVISNSPFIL NLDCDHYINN SQALRAGICF MLGRDSDTVA FVQFPQRFEG
481 VDPTDLYANH NRIFFDGTLR ALDGMQGPIY VGTGCMFRRI TLYGFDPPRI NVGGPCFPSL
541 GGMFAKTKYE KPGLELTTKA AVAKGKHGFL PLPKKSYGKS DAFVDTIPRA SHPSPFLSAD
601 EAAAIVADEA MITEAVEVCT AAYEKKTGWG SDIGWVYGTV TEDVVTGYRM HIKGWRSRYC
661 SIYPHAFIGT APINLTERLY QVLRWSTGSL EIFFSRNNPL FGSTFLHPLQ RVAYINITTY
721 PFTALFLIFY TTVPALSFVT GHFIVQRPTT MFYVYLAIVL GTLLILAVLE VKWAGVTVFE
781 WFRNGQFWMT ASCSAYLAAV CQVLVKVVFR RDISFKLTSK QPAGDEKKDP YADLYVVRWT
841 WLMVTPIIII LVNIIGSAVA FAKVLDGEWT HWLKVAGGVF FNFWVLFHLY PFAKGLLGRH
901 GKTPVVVLVW WAFTFVITAV LYINIPHIHG PGGKHGGAIG KHGAAHHGKK FDLDNLSYNW
961 P
```

The sequence of a wild type cellulose synthase-like CslF6 (CslF6) coding sequence from Miscanthus lutarioriparius provided herein as SEQ ID NO: 21.

```
    1 MAPGGGDGRR NGEAATGQQA NGGNNAKAKH GCVCGFPVCA CAGAAAVASA ASSADMDRVA
  61 VAATEGQIGA VNDESWIAVD LSDDLSGDGA DPGVALEDRP VFRTEKIKGI LLHPYRVLIF
121 VRLIAFTLFV IWRISHRNPD ALWLWVTSIA GEFWFGFSWL LDQLPKLNPI NRVPDLAVLR
181 QRFDRADGTS LLPGLDIFVT TADPFKEPIL STANSILSIL AADYPVERNT CYLSDDSGML
241 LTYEAIAEAA KFATVWVPFC RKHGIEPRGP ESYFELKSHP YIGRSQEDFV NDRRRVRKEY
301 DEFKARINGL EHDIKQRSDA YNAARGLKDG EPRATCMADG NQWEGTWVEP SENHRKGDHA
361 GIVYVLLNHP SHSRQLGPPA SADNPLDFSM VDVRLPMLVY VSREKRPGFN HEKKAGAMNA
421 LTRCSAVISN SPFILNLDCD HYINNSQALR AGICFMLGRD SDTVAFVQFP QRFEGVDPTD
481 LYANHNRIFF DGTLRALDGM QGPIYVGTGC MFRRITLYGF DPPRINVGGP CFPSLGGMFT
541 KTKYEKPGLE LTTKAAVAKG KHGFLPLPKK SYGKSDAFVD TIPRASHPSP FADADEAAAI
601 VADEATITES VAVCTAAYEK KTGWGSDIGW VYGTVTEDVV TGYRMHIKGW RSRYCSIYPH
661 AFIGTAPINL TERLYQVLRW STGSLEIFFS RNNPLFGSTF LHPLQRVAYI NITTYPFTAL
721 FLIFYTTVPA LSFVTGHFIV QRPTTMFYVY LAIVLGTLLI LAVLEVKWAG VTVFEWFRNG
781 QFWMTASCSA YLAAVCQVLV KVVFRRDISF KLTSKQPAGD EKKDPYADLY VVRWTWLMVT
841 PIIIILVNII GSAVAFAKVL DGEWTHWLKV AGGVFFNFWV LFHLYPFAKG ILGKHGKTPV
901 VVLVWWAFTF VITAVLYINI PHIHGPGGKH GGAIGKHGAA HHGKRYELYG WP
```

The sequence of a wild type cellulose synthase-like CslF6 (CslF6) coding sequence from Setaria italica provided herein as SEQ ID NO: 22.

```
    1 MAPGGGDGRR NNGEQANGNN RHGCVCGFPV CACAGAAAVA SAASSADMDR VVAVAATEGQ
  61 IGAVNDESWV AVDLSDDLSG DGGDDGVAIE DRPVFRTEKI KGVLLHPYRV LIFVRLIAFT
121 LFVIWRISHR NPDAQWLWVT SIAGEFWFGF SWLLDQLPKL NPINRVPDLA VLRQRFDRAD
181 GTSRLPGLDI FVTTADPFKE PILSTANSIL SILAADYPVE KNTCYLSDDS GMLLTYEAMV
241 EAAKFATVWV PFCRKHGIEP RGPESYFELK SHPYMGRSQE DFVNDRRRVR KEYDEFKARI
301 NGLEHDIKQR SDAYNAARGL KDGEPRATWM ADGNQWEGTW VEPSENHRKG DHTGIVLVLV
361 NHPSHGRQFG PPASADNPLD FSMVDVRLPM LVYVSREKRP GFNHEKKAGA MNALTRCSAV
421 LTNSPFILNL DCDHYINNSQ ALRAGICFML GRDSDTVAFV QFPQRFEGVD PTDLYANHNR
481 IFFDGTLRAL DGMQGPIYVG TGCLFRRVTL YGFDPPRINV GGQCFPSLGG MFAKTKYEKP
541 GLEMSTAKGA ATAVVAKGKH GFLPLPKKSY GKSEAFVDSI PRASHPSPFA NATGDAGVLT
601 DEATISEAVA VTTAAYEKKT GWGSNIGWVY GTVTEDVVTG YRMHIKGWRS RYCSIYPHAF
661 IGTAPINLTE RLYQVLRWST GSLEIFFSKN NPLFGSTFLH PLQRVAYINI TTYPFTALFL
721 IFYTTVPALS FVTGHFIVQR PTTMFYVYLA IVLGTLLILA VLEVKWAGVT VFEWFRNGQF
781 WMTASCSAYL AAVCQVVVKV VFRRDISFKL TSKQPAGDEK KDPYADLYVV RWTWLMVMPI
841 IIILVNIIGS AVAFAKVLDG EWTHWLKVAG GVFFNFWVLF HLYPFAKGIL GKHGKTPVVV
901 LVWWAFTFVI TAVLYINIPH IHGPGGKHGH GGALGKHAHG HHAGSKFGYS EVYGWP
```

The sequence of a wild type cellulose synthase-like CslF6 (CslF6) coding sequence from Dichanthelium oligosanthes provided herein as SEQ ID NO: 23.

```
    1 MAPGGGDGRR NGEAQANGKH GCACGFPVCA CAGAAAVASA ASSADMDRVA VAATEGQIGA
  61 VNDESWVAVD LSDDLSGDGA DDGVAIEDRP VFRTEKIKGI LLHPYRVLIF VRLIAFTLFV
121 IWRISHRNPD AQWLWVTSIA GEFWFGFSWL LDQLPKLNPI NRVPDLAVLR QRFDRVDGTS
181 RLPGLDIFVT TADPFKEPIL STANSILSIL AADYPVERNT CYLSDDSGML LTYEAMVEAA
241 KFATVWVPFC RKHGIEPRGP ESYFELKSHP YMGRSQEDFV NDRRRVRKEY DEFKARINGL
301 EHDIKQRSDA YNAARGLKDG EPRATWMADG SQWEGTWVEP SENHRKGDHA GIVLMLVNHP
361 SHTRQLGPPA SADNPLDFSM VDVRLPMLVY VAREKRPGFN HEKKAGAMNA LTRCSAVLTN
421 SPFILNLDCD HYINNSQALR AGICFMLGRD SDTVAFVQFP QRFEGVDPTD LYANHNRIFF
481 DGTLRALDGM QGPIYVGTGC LFRRITLYGF DPPRINVGGP CFPALGGMFA KTKYEKPGLE
541 MTTKAAVAKG KHGFLPLPKK SYGKSDAFVD SIPRASHPSP YAEAEAVVAT DEATITEAVA
601 VTTAAYEKKT GWGSSIGWVY GTVTEDVVTG YRMHIKGWRS RYCSIYPHAF IGTAPINLTE
661 RLYQVLRWST GSLEIFFSKN NPLFGSTFLH PLQRVAYINI TTYPFTALFL IFYTTVPALS
721 FVTGHFIVQR PTTMFYVYLA IVLGTLLILA VLEVKWAGVT VFEWFRNGQF WMTASCSAYL
781 AAVCQVLVKV VFRRDISFKL TSKQPSGDEK KDPYADLYVV RWTWLMVTPI IIILVNIIGS
841 AVAFAKVLDG EWTHWLKVAG GVFFNFWVLF HLYPFAKGIL GKHGKTPVVV LVWWAFTFVI
901 TAVLYINIPH IHGPGGGKHG GVLGKHAAHR GHHGGGEAYG WP
```

The sequence of a wild type cellulose synthase-like CslF6 (CslF6) coding sequence from Panicum hallii provided herein as SEQ ID NO: 24.

```
    1 MAPGGGDGRR NGEQPQQASG GRHGCACGFP VCACAGAAAV ASAASSADMD RAAVAATEGQ
  61 IGAVNDESWV AVDLSDDLSG DGGDDGVAIE DRPVFRTEKI KGVLLHPYRV LIFVRLIAFT
121 LFVIWRISHR NPDALWLWVT SIAGEFWFGF SWLLDQLPKL NPINRVPDLA VLRQRFDRAD
181 GTSRLPGLDI FVTTADPFKE PILSTANSIL SILAADYPVE KNTCYLSDDS GMLLTYEAMV
241 EAAKFATVWV PFCRKHGIEP RGPESYFELK SHPYMGRSQE DFVNDRRRVR KEYDEFKARI
301 NGLEHDIKQR SDAYNAARGL KDGEPRATWM ADGSQWEGTW VEPSENHRKG DHAGIVLVLV
361 NHPSHSRQLG PPASADNPLD FSMVDVRLPM LVYVSREKRP GFNHEKKAGA MNALTRCSAV
421 LTNSPFILNL DCDHYINNSQ ALRAGICFML GRDSDTVAFV QFPQRFEGVD PTDLYANHNR
481 IFFDGTLRAL DGMQGPIYVG TGCLFRRVTL YGFDPPRINV GGPCFPSLGG MFAKTKYEKP
541 GLEMTTKAAV AKGKHGFLPL PKKAYGKSDA FVDSIPRASH PSPFADAAAV VADEATISEA
601 VAVTTAAYEK KTGWGSNIGW VYGTVTEDVV TGYRMHIKGW RSRYCSIYPH AFIGTAPINL
661 TERLYQVLRW STGSLEIFFS RNNPLFGSTF LHPLQRVAYI NITTYPFTAL FLIFYTTVPA
721 LSFVTGHFIV QRPTTMFYVY LAIVLGTLLI LAVLEVKWAG VTVFEWERNG QFWMTASCSA
781 YLAAVCQVVV KVVFRRDISF KLTSKQPAGD EKKDPYADLY VVRWTWLMVT PIIIILVNII
841 GSAVAFAKVL DGEWTHWLKV AGGVFFNFWV LFHLYPFAKG ILGKHGKTPV VVLVWWAFTF
901 VITAVLYINI PHIHGPGGKH GHGGGMGRHA HHHAGSTHVT ELYGWP
```

The sequence of a wild type cellulose synthase-like CslF6 (CslF6) coding sequence from Panicum virgatum provided herein as SEQ ID NO: 25.

```
    1 MAWHGRVLIF VRLIAFTLFV IWRISHRNPD ALWLWVTSIA GEFWFGFSWL LDQLPKLNPI
  61 NRVPDLAVLR QRFDRADGTS RLPGLDIFVT TADPFKEPIL STANSVLSIL AADYPVERNT
121 CYLSDDSGML LTYEAMAEAA KFATVWVPFC RKHGIEPRGP ESYFELKSHP YMGRSQEDFV
181 NDRRRVRKEY DEFKARINGL EHDIKQRSDA HNAARGLKDG QPRATWMADG TQWEGTWVEP
241 AENHRKGDHA GIVQVLVNHP SHSRQLGPPA SADNPLDFSM VDVRLPMLVY VSREKRPGFN
301 HEKKAGAMNA LTRCSAVLTN SPFILNLDCD HYINNSQALR AGICFMLGRD SDTVAFVQFP
361 QRFEGVDPTD LYANHNRIFF DGTLRALDGM QGPIYVGTGC LFRRVTLYGF DPPRINVGGP
421 CFPSLGGMFA KARYEKPGLE MTTKAAVAKG KHGFLPLPRK AYGKSEAFVD SIPRASHPSP
481 FAAAAAVVAD EATISEAVAV TTAAYEKKTG WGSNIGWVYG TVTEDVVTGY RMHIKGWRSR
541 YCSIYPHAFI GTAPINLTER LYQVLRWSTG SLEIFFSRNN PLFGSTFLHP LQRVAYINIT
601 TYPFTAIFLI FYTTVPALSF VTGHFIVQRP TTMFYVYLAI VLGTLLILAV LEVKWAGVTV
661 FEWFRNGQFW MTASCSAYLA AVCQVLVKVV FRRDISFKLT SKQPAGDEKK DPYADLYVVR
721 WTWLMVTPII IILVNIVGSA VAFAKVVDGE WTHWLKVAGG VFFNFWVLFH LYPFAKGILG
781 RHGKTPVVVL VWWAFTFVIT AVLYINIPHI HGPGGKHAHG GAMGRHAHHH AGNNKFQEIY
841 GWP
```

The sequence of a wild type cellulose synthase-like CslF6 (CslF6) coding sequence from Brachypodium distachyon provided herein as SEQ ID NO: 26.

```
    1 MAPAVAGGSS RGAGCKCGFQ VCVCSGSAAV ASAGSSLEVE RAMAVTPVEG QAAPVDGESW
  61 VGVELGPDGV ETDESGAGVD DRPVFKTEKI KGVLLHPYRV LIFVRLIAFT LFVIWRISHK
121 NPDTMWLWVT SICGEFWFGF SWLLDQLPKL NPINRIPDLA VLRQRFDRAD GTSTLPGLDI
181 FVTTADPIKE PILSTANSVL SILAADYPVD RNTCYISDDS GMLMTYEAMA ESAKFATLWV
241 PFCRKHGIEP RGPESYFELK SHPYMGRAHD EFVNDRRRVR KEYDDFKAKI NSLETDIQQR
301 NDLHNAAVPQ NGDGIPRPTW MADGVQWQGT WVEPSANHRK GDHAGIVLVL IDHPSHDRLP
361 GAPASADNAL DFSGVDTRLP MLVYMSREKR PGHNHQKKAG AMNALTRASA LLSNAPFILN
421 LDCDHYINNS QALRAGICFM VGRDSDTVAF VQFPQRFEGV DPTDLYANHN RIFFDGTLRA
481 LDGMQGPIYV GTGCLFRRIT VYGFDPPRIN VGGPCFPALG GLFAKTKYEK PSMEMTMARA
541 NQAVVPAMAK GKHGFLPLPK KTYGKSDKFV DTIPRASHPS PYAAEGIRVV DSGAETLAEA
601 VKVTGSAFEQ KTGWGSELGW VYDTVTEDVV TGYRMHIKGW RSRYCSIYPH AFIGTAPINL
661 TERLFQVLRW STGSLEIFFS KNNPLFGSTY LHPLQRVAYI NITTYPFTAI FLIFYTTVPA
721 LSFVTGHFIV QRPTTMFYVY LGIVLATLLI IAVLEVKWAG VTVFEWERNG QFWMTASCSA
781 YLAAVCQVLT KVIFRRDISF KLTSKLPAGD EKKDPYADLY VVRWTPLMIT PIIIIFVNII
841 GSAVAFAKVL DGEWTHWLKV AGGVFFNFWV LFHLYPFAKG LLGKHGKTPV VVLVWWAFTF
901 VITAVLYINI PHIHGGGGKH SVGHGMHHGK KFDGYYLWP
```

The sequence of a wild type cellulose synthase-like CslF6 (CslF6) coding sequence from Avena magna provided herein as SEQ ID NO: 27.

```
    1 MAPAVAGGGR VRSNEAPAAS AAAAATGNPC ACGFQVCACT GTAAVASAAS SVDMDIMATG
  61 RIGPLNDESW VGVELGEDGE TDESGAAVDD RPVFRTEKIK AVLLYPYRVL IFVRLIAFTL
121 FVIWRISHKN PDAMWLWVTS ICGEFWFGFS WLLDQLPKLN PINRVPDLAV LRQRFDRPDG
181 TSTLPGLDIF VTTADPFKEP ILSTANSVLS ILAADYPVDR NTCYVSDDSG MLLTYEALAE
241 ASKFATLWVP FCRKHGIEPR GPESYFELKS HPYMGRAQDE FVNDRRRVRK EYDEFKARIN
301 SLDHDIRQRN DGYNAANAHR EGEPRPTWMA DGTQWEGTWV DASENHRKGD HAGIVKVLLN
361 HPSHSRQYGP PASANNPLDF SGVDVRVPML VYVSREKRPG HNHQKKAGAM NALTRASALL
421 SNAPFILNLD CDHYINNSQA LRSGICFMLG RDSDTVAFVQ FPQRFEGVDP TDLYANHNRI
481 FFDGSLRALD GMQGPIYVGT GCLFRRITVY AFDPPRINVG GPCFPMLGGM FAKTKYQKPG
541 LEMTMAKAKA APVPAKGKHG FLPLPKKTYG KSDAFVDSIP LASHPSPYVA AYNTAEGIVT
601 DEATMAEAVN VTAAAFEKKT GWGKEIGWVY DTVTEDVVTG YRMHIKGWRS RYCSIYPHAF
661 IGTAPINLTE RLFQVLRWST GSLEIFFSKN NPLFGSTYLH PLQRIAYINI TTYPFTAIFL
721 IFYTTVPALS FVTGHFIVQR PTTMFYVYLG IVLATLLIIA VLEVKWAGVT VFEWFRNGQF
781 WMTASMSAYL QAVCQVLIKV IFQKDISFKL TSKLPAGDEK KDPYADLYVV RWTPLMIVPI
841 IVIFVNIIGS AVAFAKVLDG EWTHWLKVAG GVFFNFWVLF HLYPFAKGIL GKHGKTPVVV
901 LVWWAFTFVI TAVLYINIPH MHSPGGKHTK VAHGHHGQKF LGWP
```

The sequence of a wild type cellulose synthase-like CslF6 (CslF6) coding sequence from Avena sativa provided herein as SEQ ID NO: 28.

```
    1 MAPAVAGGGR VRSNEAPAAS AAAAATGNPC ACGFQVCACT GTAAVASAAS SVDMDIMATG
  61 RIGPLNDESW VGVELGEDGE TDESGAAVDD RPVFRTEKIK AVLLYPYRVL IFVRLIAFTL
121 FVIWRISHKN PDAMWLWVTS ICGEFWFGFS WLLDQLPKLN PINRVPDLAV LRQRFDRPDG
181 TSTLPGLDIF VTTADPFKEP ILSTANSVLS ILAADYPVDR NTCYVSDDSG MLLTYEALAE
241 ASKFATLWVP FCRKHGIEPR GPESYFELKS HPYMGRAQDE FVNDRRRVRK EYDEFKARIN
301 SLDHDIRQRN DGYNAANAHR EGDPRPTWMA DGTQWEGTWV DASENHRKGD HAGIVKVLLN
361 HPSHSRQYAP ASADNPLDFS GVDVRVPMLV YVSREKRPGH NHQKKAGAMN ALTRASALLS
421 NAPFILNLDC DHYINNSQAL RSGICFMLGR DSDTVAFVQF PQRFEGVDPT DLYANHNRIF
481 FDGSLRALDG MQGPIYVGTG CLFRRITVYA FDPPRINVGG PCFPMLGGMF AKTKYQKPGL
541 EMTMAKAKAA PVPAKGKHGF LPLPKKTYGK SDAFVDSIPL ASHPSPYVAA YNTAEGIVTD
601 EATMAEAVNV TAAAFEKKTG WGKEIGWVYD TVTEDVVTGY RMHIKGWRSR YCSIYPHAFI
661 GTAPINLTER LFQVLRWSTG SLEIFFSKNN PLFGSTYLHP LQRIAYINIT TYPFTAIFLI
721 FYTTVPALSF VTGHFIVQRP TTMFYVYLGI VLATLLIIAV LEVKWAGVTV FEWFRNGQFW
781 MTASMSAYLQ AVCQVLIKVI FQKDISFKLT SKLPAGDEKK DPYADLYVVR WTPLMIVPII
841 VIFVNIIGSA VAFA
```

The sequence of a wild type cellulose synthase-like CslF6 (CslF6) coding sequence from Triticum turgidum subsp. Durum provided herein as SEQ ID NO: 29.

```
    1 MAPAVAGGGR VRSNEPAAAA ASDKPCVCGF QVCACTGSAA VASAASSLDM DIVAMGQIGA
  61 ANSVLSILAA DYPVDRNTCY VSDDSGMLLT YEALAESSKF ATLWVPFCRK HGIEPRGPES
121 YFELKSHPYM GRAQDEFVND RRRVRKEYDE FKARINSLEH DIKQRNDGYN AANAHREGEP
181 RPTWMADGTQ WEGTWVDASE NHRRGDHAGI VRVLLNHPSH RRQTGPPASA DNPLDFSGVD
241 ARLPMLVYVS REKRPGHDHQ KKAGAMNALT RASALLSNSP FILNLDCDHY INNSQALRAG
301 ICFMVGRDSD TVAFVQFPQR FEGVDPTDLY ANHNRIFFDG TLRALDGMQG PIYVGTGCLF
361 RRITVYGFDP PRINVGGPCF PRLAGLFAKT KYEKPSLEMT MAKAKAAPVP AKGKHGFLPL
421 PKKTYGKSDA FVDSIPRASH PSPYAAAAEG IVADEATIVE AVNVTAAAFE KKTGWGKEIG
481 WVYDTVTEDV VTGYRMHIKG WRSRYCSIYP HAFIGTAPIN LTERLFQVLR WSTGSLEIFF
541 SKNNPLFGST YLHPLQRVAY INITTYPFTA IFLIFYTTVP ALSFVTGHFI VQRPTTMFYV
601 YLGIVLSTLL VIAVLEVKWA GVTVFEWFRN GQFWMTASCS AYLAAVCQVL TKVIFRRDIS
661 FKLTSKLPSG DEKKDPYADL YVVRWTPLMI TPIIIIFVNI IGSAVAFAKV LDGEWTHWLK
721 VAGGVFFNFW VLFHLYPFAK GILGKHGKTP VVVLVWWAFT FVITAVLYIN IPHMHSSGGK
781 HTTVHGHHGK KFVDAGYYNW P
```

### Cereal Plant Carrying a Mutation in the CslF6 Gene

The present invention provides a cereal plant or a part thereof, wherein the plant or part thereof has an altered (1,3;1,4)-β-glucan content, wherein said plant or part thereof carries a mutation in the CslF6 gene and wherein said mutated CslF6 gene encodes a mutant CslF6 polypeptide. The mutation in the CslF6 gene may be any of the mutations described herein (including one or more mutation and/or more than one type of mutation), however in some embodiments of the invention the mutation is a point mutation in the coding region, such as the switch motif (SEQ ID NO: 14), of the CslF6 gene.

The mutant CslF6 polypeptide encoded by said mutant CslF6 gene may be any mutant CslF6 polypeptide. In some embodiment, the mutant CslF6 polypeptide contain one amino acid substitution. In particular, the mutant CslF6 polypeptide may comprise a substitution of one amino acid switch motif of CslF6. The mutant CslF6 polypeptide may comprise one or more of the following substitutions of a non-polar amino acid to a charged amino acid; and substitution of a polar amino acid to a non-polar amino acid.

As used herein the terms “substitution of amino acid XX for amino acid YY” or “substitution of amino acid XX to amino acid YY” refers to amino acid XX in a reference sequence (typically the CslF6 wild type sequence) being replaced by amino acid YY. In one embodiment the plant may contain a T to H mutation in the switch motif.

### Plant Products

The invention also provides plant products prepared from a barley plant having an altered (1,3;1,4)-β-glucan content and carrying a mutation in the CslF6 gene, or a progeny thereof, wherein said mutated CslF6 gene encodes a mutant CslF6 polypeptide.

The plant product may be any product prepared from a barley plant, for example a food, a feed (for livestock) or a beverage, such a flour, syrup, malt, beer, dough, cake, or bread. For example, he food product may be a bagel, a biscuit, a bread, a bun, a croissant a dumpling, an English muffin, a muffin, a pita bread, a quick bread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut an egg roll, an extruded snack, a fruit and grain bar, a microwaveable snack product a nutritional bar, a pancake, a par-baked bakery product, a pretzel, a pudding, a granola-based product, a snack chip, a snack food, a snack mix, a waffle, a pizza crust, animal food or pet food.

### Treatment

Whilst the invention may be particularly useful in the treatment or prophylaxis of humans, it is to be understood that the invention is also applicable to non-human subjects including but not limited to agricultural animals such as cows, sheep, pigs, poultry such as chickens and the like, domestic animals such as dogs or eats, laboratory animals such as rabbits or rodents such as mice, rate, hamsters, or animals that might be used for sport such as horses. The method of treating the subject, particularly humans, may comprise the step of administering altered wheat grain, flour, starch, isolated β-glucan or a composition comprising β-glucan, or a food or drink product as defined herein to the subject, in one or more doses, in an amount and for a period of time whereby a physiological parameter is modified. For example, the level of cholesterol uptake in the large intestine of the subject is reduced, which leads to decreased cholesterol levels in fee bloodstream of the subject. Dosages may vary depending on the condition being treated or prevented but are envisaged for humans as being the β-glucan in at least 1 g of wheat grain or flour of the invention per day, including at least 2 g per day, including at least 10 g or at least 20 g per day. Administration of greater than about 100 grams of grain or flour per day may require considerable volumes of delivery and reduce compliance. The dosage for a human can be between 0.2 g and 5 g of including, which may be in the form of a food product containing grain or flour of the invention, which is equivalent to between about 5 g and about 60 g of wheat grain or flour per day (or other cereal plant), or for adults between about 5 g and 100 g/per day. It will be understood that one benefit of the present invention is that it provides for products such as bread that are of particular nutritional benefit, and moreover it does so without the need to post-harvest modify the constituents of the harvested/processed plant.

### Example I

### Materials and Methods

### Expression and Purification of HvCslF6 in SF9 Insect Cells

The barley cellulose synthase-like F6 gene (HvCslF6) encoding an N-terminal 12×His tag was amplified from the HvCslF6-pCR8 construct and cloned into NotI and HindIII restriction sites of the baculovirus vector pACEBac1 using the primers listed. The mutants of HvCslF6 (D629G, I757L, Y787H and CslF6-CesA8-sm) and PttCesA8 (H832Y, CesA8-CslF6-sm) were generated using the mutagenesis primers listed.

| | | Sequence 5′-3′ |
|---|---|---|
| Wild | Forward | ACAATAAGCGGCCGCATGCACCAT |
| type | | CACCATCACCATCACCATCACCAT |
| HvCslF6 | | CACCATATGCACCATCACCATCAC |
| | | CATCAC |
| | | (SEQ ID NO: 1) |
| | Reverse | TATAACGAAAGCTTTCAATGGAGC |
| | | CAGCCATAGAGCCCTG |
| | | (SEQ ID NO: 2) |
| CslF6- | Forward | GAGATCGGCTGGGTGTACGGCACC |
| D629G | | GTCACGGAGGACGTG |
| | | (SEQ ID NO: 3) |
| | Reverse | CTATCCACGCTGCTCGTCCTCGCC |
| | | GTGCTGGAGGTC |
| | | (SEQ ID NO: 65) |
| CsIF6- | Forward | CACGTCCTCCGTGACGGTGCCGTA |
| I757L | | CACCCAGCCGATCTC |
| | | (SEQ ID NO: 4) |
| | Reverse | GACCTCCAGCACGGCGAGGACGAG |
| | | CAGCGTGGATAG |
| | | (SEQ ID NO: 66) |
| CslF6- | Forward | TGTTCTGCTCACCTGGCTGCAGTG |
| Y787H | | TGCCAGGTGCTGACCAAGGTG |
| | | (SEQ ID NO: 5) |
| | Reverse | TGCAGCCAGGTGAGCAGAACAGGA |
| | | TGCTGTCATCCAGAACTGGCC |
| | | (SEQ ID NO: 6) |
| CslF6- | Forward | ATCGGTGGCGTGAGCGCTCACCTC |
| CesA8- | | GCCGCCGTCTGCCAGGTGCTG |
| sm | | (SEQ ID NO: 7) |
| | Reverse | AGCGCTCACGCCACCGATCACCCA |
| | | GAACTGGCCGTTCCTGAACCA |
| | | (SEQ ID NO: 8) |
| CesA8- | Forward | GTGTCCGCTTACCTGTTCGCTGTG |
| H832Y | | TTCCAGGGATTCTTGAAAATG |
| | | (SEQ ID NO: 9) |
| | Reverse | AGCGAACAGGTAAGCGGACACGCC |
| | | TCCGATCACCCAGAACTGTTC |
| | | (SEQ ID NO: 10) |
| CesA8- | Forward | ACCGCTTCCTGCAGCGCTTACCTC |
| CslF6- | | TTTGCCGTCTTCCAGGGATTC |
| sm | | (SEQ ID NO: 11) |
| | Reverse | AGCGCTGCAGGAAGCGGTCATCCA |
| | | GAACTGTTCATTACGCCACAA |
| | | (SEQ ID NO: 12) |

Expression of HvCslF6 and its mutants was performed in Sf9 insect cells and purification of the protein variants was carried out as previously described for the hybrid aspen (Populus tremula x tremuloides, Ptt) CesA8 (21) except 1 mM TCEP [tris(2-carboxyethyl)phosphine] instead of β-mercaptoethanol was used as a reducing agent in the buffer. PttCesA8 was purified using the same procedure as for HvCslF6, but with a modified buffer system which contained 20 mM Tris pH 7.5, 100 mM NaCl, 5 mM sodium phosphate and 5 mM sodium citrate. After gel filtration chromatography, the PttCesA8 trimeric fractions were used for activity assays.

### Activity Assays

Activity assays were performed as described (22, 27). Briefly, for the initial metal dependent assay, 5 μM HvCslF6 was incubated at 30° C. with 5 mM UDP-glucose (UDP-Glc), and 0.25 μCi UDP-[3H]-Glc in 20 mM of EDTA, MgCl2, MnCl2 or CaCl2 in a buffer containing 20 mM Tris (pH 7.5), 0.1M NaCl and 1 mM TCEP. The time-course of (1,3;1,4)-β-glucan biosynthesis was monitored in different time intervals up to 24 h. For enzymatic degradation, the in vitro product obtained after an overnight synthesis reaction was hydrolyzed with 5 U of endo-(1,3)-β-glucosidase from Trichoderma sp. or endo-(1,4)-β-glucanase from Trichoderma longibrachiatum or Lichenase endo-(1,3;1,4)-β-glucanase from Bacillus subtilis (Megazyme, Wicklow, Ireland). The endo-glucanases were added at the beginning of the synthesis reaction and incubation continued for overnight. The synthesis reaction was spotted onto Whatman 3 MM paper and developed by descending chromatography in 60% ethanol as described (23). Following paper chromatography, the polymeric product was quantified by scintillation counting. Non-hydrolyzed samples for this analysis were treated similarly without addition of any endo-glucanases.

### HPLC Analysis of Oligosaccharides Derived From Hv CslF6-Synthesized (1,3;1,4)-β-Glucan

The in vitro (1,3;1,4)-β-glucan polymeric product was synthesized overnight under the experimental conditions described above, except that 1 mg/ml of Hv CslF6 and 10 mM U-DPGlc substrate without UDP-[3H]-Glc was used. After the synthesis, the entire reaction mixture was loaded onto a 3-kDa MWCO Amicon Ultra 0.5 mL filter to remove small molecules such as excess UDP-Glc, oligosaccharides and detergent. The retained concentrated protein and polymeric material was washed thrice with endo-glucanase-specific buffers: 100 mM sodium acetate buffer pH 4.5 for endo-1,3-β-glucosidase and endo-1,4-β-glucosidase and 100 mM sodium phosphate buffer pH 6.5 for lichenase. The retained polymeric product was hydrolyzed with 5 U of each endo-glucanase at 40° C. for 3 hours. After enzymatic hydrolysis of the polymer, the mixtures were loaded again onto a new 3-kDa MWCO Amicon Ultra 0.5 mL filters. The filtrates containing oligosaccharide products were analyzed by HPLC.

For the control experiment, commercial barley (1,3;1,4)-β-glucan (Sigma-Aldrich, St. Louis, USA), 0.25 mg/ml was suspended in the endo-(1,3)-β-glucosidase- and lichenase-specific buffers. The polymer was hydrolyzed with each enzyme and the oligosaccharides released were analyzed by HPLC.

The (1,3;1,4)-β-glucan derived oligosaccharides were separated on an Agilent 1260 Infinity II LC system (Agilent technologies, Santa Clara, USA) on a TSKgel column G2000PW (60 cm×7.5 mm ID) (TOSOH Biosciences) equilibrated with water. Oligosaccharides were eluted in water at a flow rate of 1 ml/min, and the elution was monitored using a refractive index detector (RID). The chromatograms were compared with that of standard cellulose oligosaccharides ranging from glucose to cellohexaose (Megazyme, Wicklow, Ireland).

### Glycosidic Linkage Analysis

Syntheses of the in vitro β-glucan polymer from both CslF6 and CesA8 were performed as described above. After synthesis, the products were precipitated with 80% v/v ethanol for 5 hours at room temperature (25° C.). The precipitates were collected by centrifugation at 21,200 g for 20 min. The pellet was washed twice with 80% v/v ethanol for 10 min at 21,200 g and air dried before glycosidic linkage analysis.

Glycosidic linkages were identified and quantified as described (41), with minor modifications. Briefly, 1 mg of the freeze-dried samples were methylated in anhydrous dimethyl sulfoxide (DMSO) by adding 0.1 mL of methyl iodide under nitrogen and sonicating for 10 min at room temperature. This step was repeated four times to avoid under-methylation of the polysaccharides. One mL of dichloromethane (DCM) was subsequently added to the samples and the methylated polysaccharides were extracted by phase partitioning against deionized water. This step was repeated three times and the resultant combined DCM phases were evaporated under a stream of nitrogen, followed by hydrolysis at 100° C. for 3 h in 1 mL of 2 M trifluoroacetic acid (TFA) under nitrogen. The hydrolysates were reduced overnight at room temperature in the presence of NaBD4 under nitrogen and acetylated with acetic anhydride at 100° C. for 12 h. The partially methylated alditol acetates (PMAAs) were recovered by evaporating the acetic anhydride solvent under a gentle stream of nitrogen and redissolving the samples in DCM. The PMAAs were purified by partitioning against deionized water and the DCM phase was transferred to a GC vial and analyzed on an Agilent 7890B/5977B GC-MS (Agilent Technologies, Santa Clara, USA) fitted with a VF-23 ms capillary column (30 m×0.25 mm, 0.25 μm, Agilent Technologies, USA). Helium was used as carrier gas and the oven temperature was programmed as follows: from 165° C. to 175° C. at 1° C./min; from 175° C. to 195° C. at 0.5° C./min; from 195° C. to 210° C. at 2° C./min, and from 210° C. to 250° C. at 10° C./min, followed by a plateau at 250° C. for 6.5 min (total run time 68 min). The fragmentation patterns of the different PMAAs were interpreted by referring to the CCRC Spectral Database for PMAAs (https://glygen.ccrc.uga.edu/ccrc/specdb/ms/pmaa/pframe.html).

### Cryo-Grids Preparation

The quality of the purified HvCslF6 protein was checked by negative staining. Immediately after gel filtration chromatography, 4 μl of protein at 0.01 mg/ml was applied to a carbon/formvar grid, followed by 2 washes with 4 μl H2O and staining with 4 μl 0.75% uranyl formate. The negatively stained grids were checked on a Fei Tecnai F20 at the Macromolecular Electron Microscopy Core (MEMC) facility at the University of Virginia. The remaining of the sample corresponding to the selected protein preparation was concentrated to ˜3 mg/ml and applied (2.5 μl) to a C-flat 300 mesh 1.2/1.3 copper grid (Electron Microscopy Sciences) glow-discharged in the presence of amylamine at 25 mA for 45 s. The grid was blotted with a Vitrobot Mark IV (FEI, Thermo Fisher Scientific, Waltham, USA) with force 7 for 12-14 s at 4° C., 100% humidity, and frozen in liquid ethane and stored in liquid nitrogen.

### Cryo-EM Data Collection

The cryo grids were screened on a Fei Titan Krios microscope operated at 300 keV and equipped with a Gatan K3 direct electron detector positioned post a Gatan Quantum energy filter at the MEMC at the University of Virginia. Datasets were collected at the Pacific Northwest Center (PNCC) on a Titan Krios microscope operated at 300 keV and equipped with a Gatan K3 direct electron detector positioned post a Gatan Quantum energy filter. Movies (6528) with 50 frames were collected from one grid with a pixel size of 0.83 Å, defocus of −2.0 μm to −1 μm with step size of 0.2 μm. The total dose was 53 e-/Å2.

### Data Processing

The cryo-EM images were processed in cryoSPARC 2.12.4 (25). Movies were corrected for full frame as well as sample deformation using Patch Motion Correction followed by full frame CTF Estimation. After CTF estimation, micrographs were curated based on estimated resolution (0 to 4.0 Å), drift (0 to 30 pixel), astigmatism (0 to 1000 Å) and defocus (0 to −3.0 μm), as well as manually inspected for outliers and ice contamination.

Particles were automatically picked using cryoSPARC's ‘Blob Picker’ with minimum and maximum particle diameters of 80 and 300 Å, respectively, extracted at a box size of 400 pixels, followed by 2D classification. Good class averages with different views were selected for template-based particle picking using a diameter of 300 Å and a minimum separation distance of 150 Å (0.5×particle diameter). The particles were extracted, and Fourier cropped to a box size of 100 pixels followed by several rounds of 2D classification with 100 to 50 classes each and a batch size of 300 per class.

An initial model was built in cryoSPARC followed by 3D-sorting. Particles belonging to the best class were re-extracted at a physical pixel size of 0.83 Å and 300-pixel box size followed by non-uniform (NU) refinement, yielding a density map with an estimated resolution of 5.5 Å based on FSC. Local refinement using a mask covering only the protein part improved the density quality significantly and yielded a map with an estimated resolution of 4.0 Å based on FSC. This map was used for model building.

The CslF6 model was built using an AlphaFold2 generated template and manually corrected in Coot (42) and refined in Phenix: refine (43).

EM and model stats
Cryo-electron microscopy data collection and processing

| | |
|---|---|
| Microscope | FEI Titan Krios |
| Voltage (keV) | 300 |
| Camera | Gatan K3 |
| Energy Filter | Gatan Quantum-LS (GIF) |
| Pixel size (Å) | 0.83 |
| Defocus range (μm) | −2.25 to −1.0 |
| Magnification (nominal) | 105,000 |
| Electron exposure (e⁻/pix/s) | 13.6 |
| Exposure rate (e⁻/Å²) | 53 |
| Frames per movie | 50 |
| Energy filter slit width (eV) | 20 |
| Automation software | SerialEM |
| Micrographs used | 4872 |
| Extracted particles | 1,544,364 |
| Particles in final 3D refinement | 80347 |
| Local resolution range (Å) | 20-4.0 |
| Resolution No mask (Å) | 6.1 |
| Resolution Spherical | 5.4 |
| Resolution Loose | 4.5 |
| Resolution Tight | 3.9 |
| Resolution Corrected | 4.0 |
| Sharpening B-factor (Å2) | −127.4 |
| EMDB ID | |

Coordinate Refinement and Validation

| | |
|---|---|
| Refinement program | Phenix (Real-space refinement) |
| Number of protein atoms (non-H) | 34713 |
| Number of ligands | CE5: 3 |
| RMSD bond (Å) | 0.007 |
| RMSD angle (°) | 1.004 |
| Ramachandran favored (%) | 91.35 |
| Ramachandran allowed (%) | 7.57 |
| Ramachandran outlier (%) | 1.08 |
| All-atom clash score | 12.9 |
| MolProbity Score | 2.13 |

B-factors (min/max/mean)

| | |
|---|---|
| Protein | 60.03/199.36/109.28 |

Overall correlation coefficient

| | |
|---|---|
| CC (mask) | 0.77 |
| CC (box) | 0.66 |
| CC (peaks) | 0.6 |
| CC (volume) | 0.75 |
| Mean CC for ligands | 0.67 |
| PDB ID | |

### Results

The approach established for hybrid aspen CesA8 (21) was adapted to express and purify barley CslF6, as described in the Methods. To facilitate the detection and purification of the recombinant enzyme, a poly-histidine tag was engineered at the protein's N-terminus.

### CslF6 Purifies as a Monomeric, Catalytically Active Species

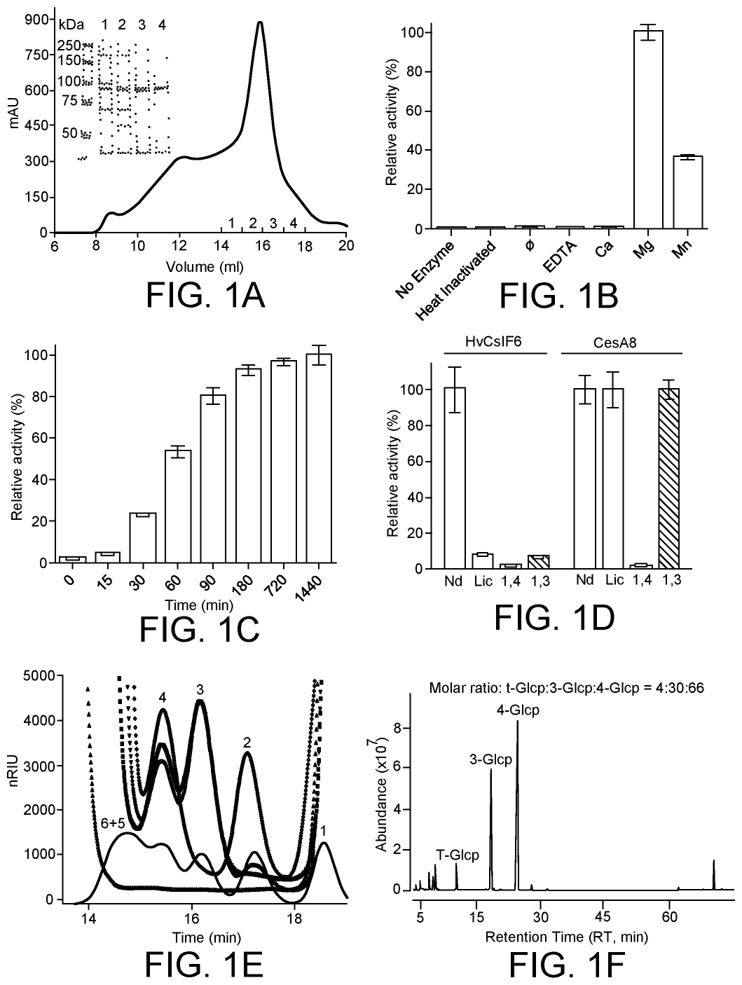

CslF6 was extracted from the membrane fraction in a detergent mixture of lauryl maltose neopentyl glycol and cholesteryl hemisuccinate (LMNG/CHS), and later exchanged into glyco-diosgenin (GDN) during metal affinity chromatography. The protein elutes as a symmetric peak from a superose 6 size exclusion chromatography column (FIG. 1A), although minor higher molecular weight species are present. Although protein molecular weights are difficult to estimate in a micelle-embedded state, the observed CslF6 elution volume suggests a lower molecular weight compared to a CesA8 homo-trimer purified in the same detergent (22).

Attempts to verify the formation of homo-oligomeric complexes by co-purification of His- and Myc-tagged CslF6 species failed. Therefore, we conclude that the higher molecular weight CslF6 species arise from co-purifying protein-bound polysaccharides of various lengths, as confirmed by cryo-EM (see below).

Catalytic activity of CslF6 can be monitored in vitro by either quantifying the produced glucan using a radiotracer or detecting the released UDP enzymatically (22, 23). CslF6's in vitro catalytic activity was tested by measuring incorporation of ³H-labeled glucose into the synthesized polymer. The labeled glucans were separated from un-polymerized tracers by descending paper chromatography, followed by scintillation counting, as previously described for the hybrid aspen CesA8 (23).

In vitro, CslF6 produces a glucan in the presence of magnesium cations and UDP-glucose (FIG. 1B). Catalytic activity is reduced to approximately 30% when magnesium is replaced with manganese, and no detectable product is formed in the presence of EDTA or calcium. Most product accumulates over ˜90 min and synthesis plateaus approximately 3 hours after initiation (FIG. 1C).

To test whether the synthesized product indeed represents a polymer harboring both (1,3)- and (1,4)-β-linked glucosyl units, the 3H-labeled polymer was hydrolyzed with enzymes that specifically degrade (1,3;1,4)-β-glucans (a lichenase), cellulose [a (1,4)-β-glucanase], or curdlan [a (1,3)-β-glucanase] (FIG. 1D). Incubating the polymer synthesized in vitro with Bacillus subtilis lichenase (24) reduces the amount of detectable polymeric product to about 20% compared to a control in the absence of hydrolase, demonstrating that the polymer is indeed a (1,3;1,4)-β-glucan (7). As expected, incubations with (1,3)-β- and (1,4)-β-glucan endohydrolases also degrade the polymer. Stretches of two or three adjacent (1,4)-linked glucosyl units are sufficiently long for degradation by (1,4)-β-glucan endohydrolases (i.e., cellulase). Cleavage by the Trichoderma sp. (1,3)-β-glucan endohydrolase also indicates the presence of (1,3)-linkages as this enzyme is able to hydrolyze either a (1,3)- or a (1,4)-β-linkage, provided that the target linkage is adjacent to a (1,3)-β-linked glucosyl unit, towards the reducing terminus of the polysaccharide (7).

### Lichenase Digestion Releases DP3 and DP4 Oligosaccharides From (1,3:1,4)-β-Glucan

Lichenase degradation of (1,3:1,4)-β-glucans releases DP3 and DP4 fragments due to the specific cleavage of (1,4)-linkages following a (1,3)-linked glucosyl unit (9). Accordingly, lichenase degradation of the CslF6-synthesized glucan produces DP3 and DP4 fragments that are indistinguishable from those released from control barley grain (1,3:1,4)-β-glucans, where CslF6 is the predominant (1,3:1,4)-β-glucan synthase (FIG. 1E). In both cases, the oligosaccharides elute from a TSKgel HPLC size exclusion chromatography column at 15.4 (DP4) and 16.1 (DP3) min. Under the same conditions, cellotetraose and cellotriose standards elute at 15.2 and 16.0 min, respectively (FIG. 1E).

For comparison, commercially available barley (1,3:1,4)-β-glucans were degraded with the lichenase, cellulase, and (1,3)-β-glucan endohydrolase used to analyze the glucan synthesized in vitro (FIG. 1). In this case, lichenase treatment generates the characteristic DP3 and DP4 fragments, cellulase releases primarily di- and tetrasaccharides, and (1,3)-β-endohydrolase treatment produces DP4, DP3 and DP2 oligosaccharides.

### Barley CslF6 Synthesizes a Polymeric (1,3:1,4)-β-Glucan

Permethylation linkage analysis confirmed the in vitro synthesis of a (1,3:1,4)-β-glucan. The method readily detects (1,3)- and (1,4)-linked glucosyl units in control barley grain polymers at an approximate ratio of 1:2.2, consistent with previous reports (24). In bona-fide cellulose, however, only 4-linked glucosyl residues are detected, in agreement with its structure.

The glucan produced by CslF6 indeed contains (1,3)- and (1,4)-linked glucosyl units at an approximate 1:2 ratio (FIG. 1F). Based on the abundance of terminal glucosyl units in the GC chromatogram, an overall length of the polysaccharide is estimated of about 25 glucosyl units. Analyzing material produced from proteoliposome-reconstituted CslF6, instead of detergent-solubilized enzyme, did not change the ratio of (1,3)- and (1,4)-linked glucosyl units, but increased the overall polymer length to about 33 residues. Thus, the glycosidic linkage analysis independently confirms the in vitro formation of a polymeric (1,3:1,4)-β-glucan by barley CslF6.

### Monomeric CslF6 Adopts a Cellulose Synthase-Like Fold

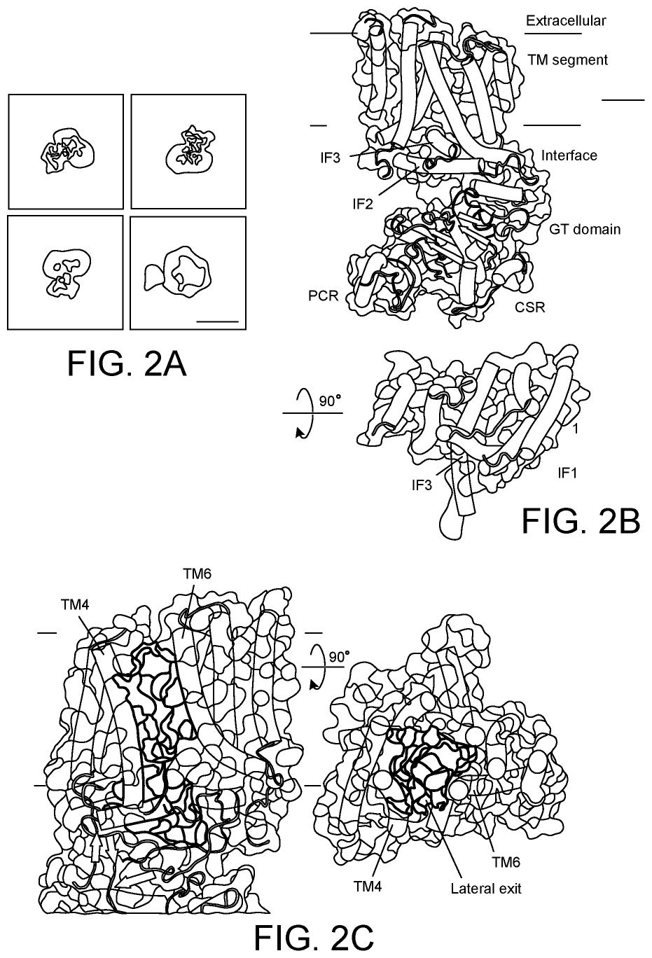

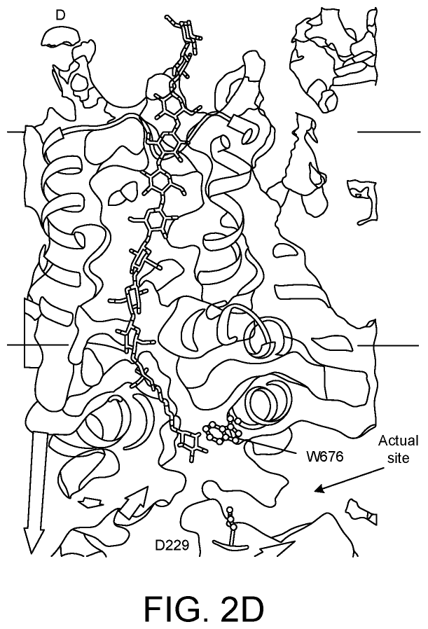

Cryo-EM analyses of CslF6 were performed with protein species eluting from a size exclusion chromatography column at higher and lower apparent molecular weights (FIG. 1A). However, in all cases, only monomeric particles were evident from 2 and 3-dimensional classifications (FIG. 2A).

Cryo-EM data processing in cryoSPARC (25) or Relion (26) generated a medium resolution map at about ˜4.0 Å resolution that resolved all of CslF6's TM helices as well as most regions of its cytosolic GT domain, (FIG. 2B). The N-terminal 92 residues and residues 518-572 of the GT domain are disordered, as also observed in CesA structures (22, 27). Building the CslF6 model took advantage of its similarity with hybrid aspen CesA8 as well as a predicted AlphaFold2 model (28, 29). Therefore, the structure represents an AlphaFold2-generated CslF6 model refined against a medium resolution cryo-EM map.

CslF6 contains seven TM helices with the C-terminus residing on the luminal side and the catalytic domain between TM helices 2 and 3 on the cytosolic side of the membrane (FIG. 2B). TM helix 7, implicated in trimer formation in hybrid aspen CesA8, packs against TM helices 5 and 6 at the periphery of the CslF6 core structure. The TM helices interact with three amphipathic cytosolic helices that run parallel to the membrane plane. The catalytic GT domain packs against these interface helices and protrudes into the cytosol by about 35 Å.

Compared to bacterial cellulose synthase and other GT2 enzymes adopting a GT-A fold (28, 30), the catalytic cores of CesA and CslF6 contain two insertions, referred to as plant conserved (PCR) and class specific (CSR) regions, respectively (31). The PCR domain, corresponding to residues 245-390, consists of two antiparallel α-helices that interact with the catalytic core on its membrane distal side, as described for CesA (FIG. 2B) (22, 32). In CesA trimers, the PCR stabilizes a trimeric assembly by forming a triangular oligomerization domain. In CslF6, the loop connecting the two PCR helices is 15 residues longer compared to hybrid aspen CesA8 and folds back onto the PCR helices.

The CslF6 CSR, corresponding to residues 514 to 618, is mostly disordered, as also observed in bona-fide CesA enzymes (22, 27, 33). Considering its proximity to the predicted water-lipid interface, the domain may interact with membrane lipids and/or other binding partners (FIG. 2B).

### CslF6 Accommodates a Nascent Glucan Chain Inside its TM Channel

CslF6 couples polysaccharide synthesis with the translocation of the polymer across the membrane. Its seven TM helices form a continuous TM channel containing a large, likely lipid-filled, lateral opening towards the bilayer phase (FIG. 2C). At lower contour levels, ribbon-shaped density is evident inside the TM channel, starting at the active side and spanning approximately half the channel (FIG. 2D). The shape and position of this non-proteinaceous density resembles the nascent cellulose polymer observed in plant and bacterial CesAs (22, 27, 30), thus likely representing a nascent (1,3:1,4)-β-glucan produced during recombinant protein expression.

Although at lower resolution and significantly weaker than the surrounding protein map, the polymer density suggests the presence of at least six glucosyl units. Notably, the glucan starts within the catalytic pocket, at the membrane distal side of Trp676 of the conserved QxxRW motif. The Trp residue of the QxxRW motif coordinates the acceptor glucosyl unit via CH-□ stacking interactions (22, 27, 30). This suggests that the oligosaccharide co-purifying with CslF6 is in a pre-translocation position, i.e. representing a state after chain elongation yet before translocation, as also observed for bacterial CesA (30).

### CslF6 Shares a Conserved Active Site With Bona Fide CesAs

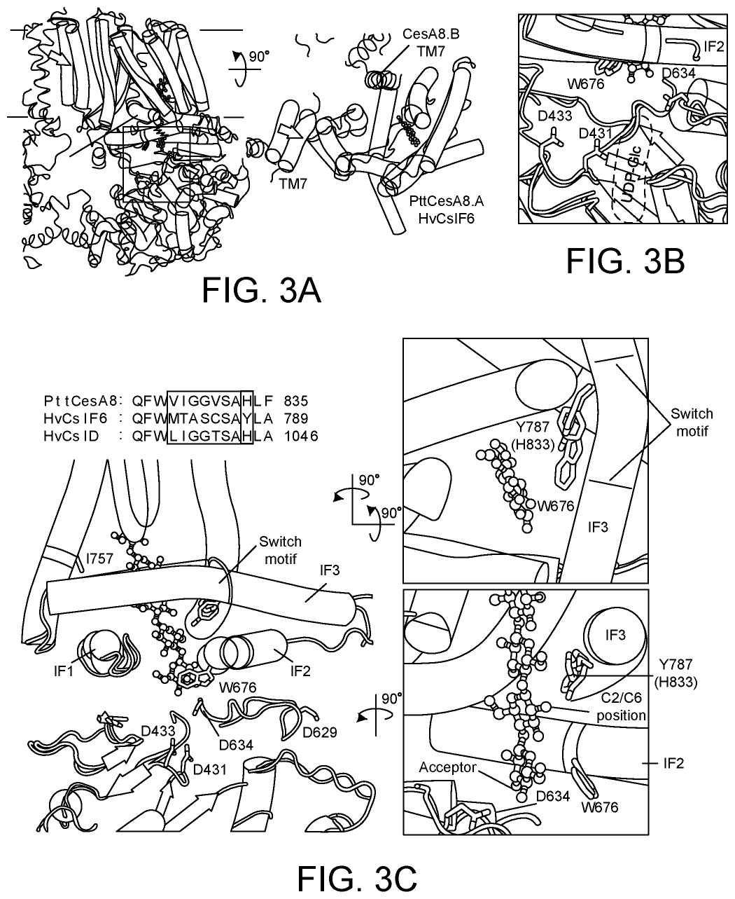

Compared to hybrid aspen CesA8, TM helix 7 of CslF6 is shifted by about 15 Å towards TM helices 5 and 6 (FIG. 3A). The corresponding helix in the CesA8 homo-trimer interacts with TM helices 4 and 5 of a neighboring protomer, thereby contributing to oligomerization (22).

As confirmed by biochemical analyses, CslF6 catalyzes glycosyl transfer to either the C3 or C4 hydroxyl of the accepting sugar, requiring proper acceptor positioning relative to a base catalyst. Analogous to bacterial and plant CesAs, the general base is likely formed by the aspartate (D634) of the conserved TED motif at the N-terminus of a short helix directly adjacent to the acceptor binding site (30).

Superimposing the CslF6 and CesA8 structures reveals a striking conservation of the catalytic pocket, including the TED motif (FIG. 3B). No additional residues or substitutions are observed that may function as base catalysts under physiological conditions or suggest an alternative substrate-binding pose. This indicates that CslF6 always binds its substrate in the same conformation and uses the same base catalyst to activate the acceptor's C3 or C4 hydroxyl group.

Focusing on the proximal region of the TM channel, the primary sequences of CesAs, CslF6s, as well as CslDs, the latter being the most closely related Csl enzymes were compared to CslF6. Strikingly, this analysis revealed a sequence motif conserved among the cellulose-producing enzymes (CesAs and CslDs) that diverged in the CslF6 lineage (FIG. 3C and FIG. 6). This ‘switch-motif’, VIGGVSAH (SEQ ID NO: 13) in CesA/CslD and MTASCSAY (SEQ ID NO: 14) in CslF6, belongs to IF3 that, together with IF1, IF2, and TM helix 3, frames the cytosolic entrance to CslF6's TM channel (FIG. 2). The CesA8 structure containing a nascent cellulose oligosaccharide reveals close proximity between the switch motif's C-terminal His832 and the second and third glucosyl units of the nascent cellulose chain (22). This residue is a tyrosine in CslF6 extending farther into the TM channel and likely forming a hydrogen bond with the translocating glucan, especially its C6 hydroxyl groups (FIG. 3C).

### The Switch Motif Controls (1,3)-β-Glycosyl Linkage Formation

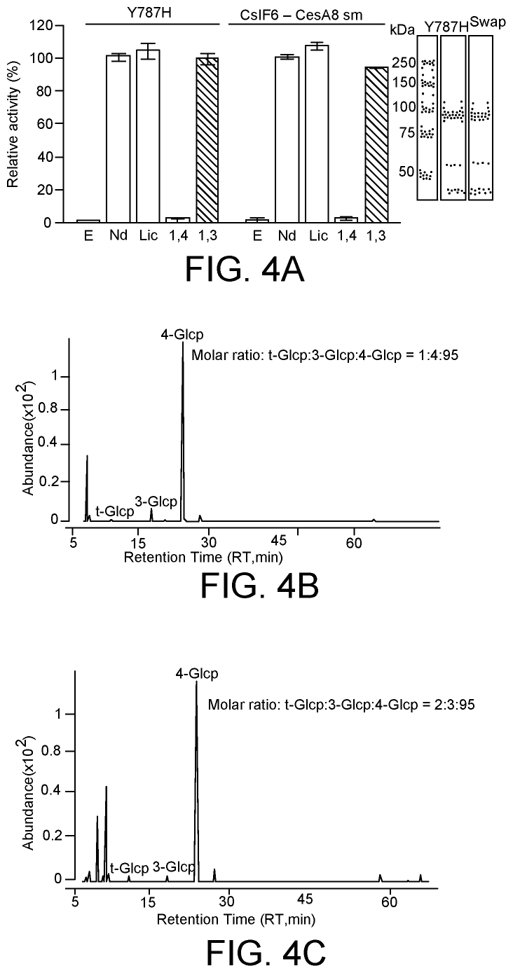

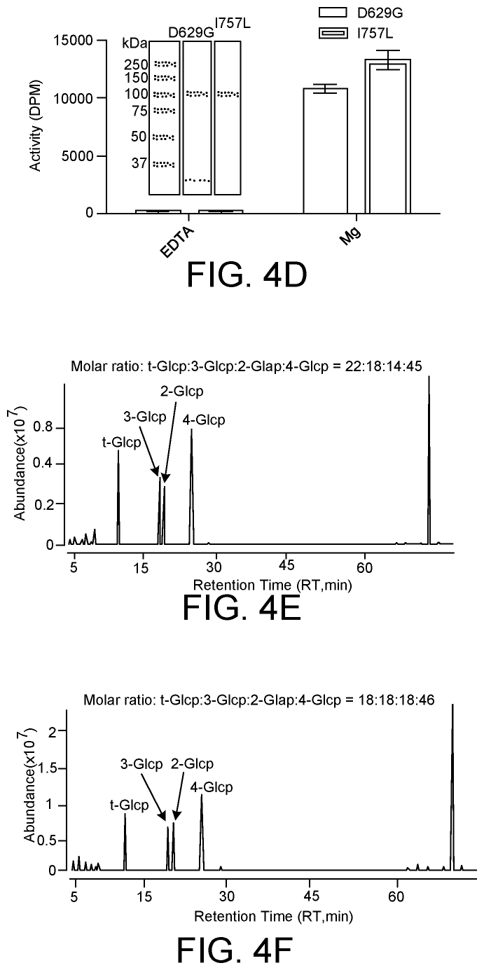

Single point mutagenesis and swapping analyses was performed to test whether the switch motif and in particular its terminal Tyr residue controls CslF6's ability to introduce (1,3)-β-glucosyl linkages into the synthesized glucan. The mutant enzymes were expressed and purified as described for wild type CslF6, with no detectable reduction in protein stability or catalytic activity (FIG. 4).

Replacing Tyr787 in barley CslF6 with the corresponding His residue found in CesAs dramatically reduces (1,3)-β-linkage formation (FIGS. 4A and B). The polymer synthesized in vitro is no longer degraded by lichenase or (1,3)-β-glucan endohydrolase, while cellulase continues to hydrolyze it. Similar results were obtained with a motif-swapped CslF6 in which the entire switch-motif was replaced with the corresponding sequence of CesAs (FIGS. 4A and C). The reverse experiment, introducing the CslF6 switch-motif into hybrid aspen CesA8, did not result in (1,3)-β-linkage formation, suggesting that the identified motif is necessary but not sufficient for (1,3)-β-glucosyl linkage formation (see discussion).

The products of the mutant enzymes were also characterized by glycosidic linkage analyses (FIGS. 4B and C). In agreement with enzymatic degradation assays, the CslF6-Y787H point mutant and switch-motif swapped constructs synthesize a predominantly (1,4)-linked β-glucan (i.e., cellulose), with only a negligible fraction of (1,3)-β-linked glucosyl units. The apparent ratio of (1,3)- to (1,4)-linkages drops from 1:2 for the wild-type enzyme to about 1:24 and 1:32 for the single residue and switch motif-swapped constructs, respectively. These mutants appear to synthesize polysaccharides with DPs close to 100 (FIGS. 4B and C). Linkage analyses did not detect any (1,3)-linkages in the products of the corresponding CesA8 mutants, in agreement with the enzymatic degradation results.

In addition to the identified switch motif, previous site-directed mutageneses of CslF6 expressed in Nicotiana benthamiana identified several residues within the enzyme's GT domain and TM region affecting the likelihood of forming (1,3)-β-glucosyl linkages (21, 34). These include an isoleucine to leucine substitution within TM helix 4 as well as an aspartate to glycine replacement within the GT domain. These mutations were introduced into wild-type CslF6 (I757L and D629G) and analyzed in vitro synthesized glucan enzymatically and via glycosidic linkage analyses. The substitutions slightly reduce (1,3)-linkage formation by CslF6, resulting in a DP3:DP4 ratio of about 1:2.5, compared to 1:2 for the wild-type enzyme (FIG. 4D-F). However, compared to the switch motif substitutions, the observed effects are minor.

### Discussion

Cellulose synthase-like enzymes synthesize diverse cell wall polysaccharides, including mannans (family A), xyloglucans (family G), cellulose (family D), as well as (1,3:1,4)-β-glucans (families F, H, and J) (35-37). On a molecular level, the enzymes differ primarily in the length and sequence of their N-terminal domains preceding the first TM helix as well as the PCR and CSR regions. It is shown here that barley CslF6 is nedded and sufficient to synthesize a (1,3;1,4)-β-glucan in vitro, confirming that the enzyme indeed introduces (1,3)-β-glucosyl linkages at irregular yet non-random positions in the otherwise (1,4)-β-linked glucan. Biosynthesis only requires the substrate UDP-glucose and magnesium as the divalent cation.

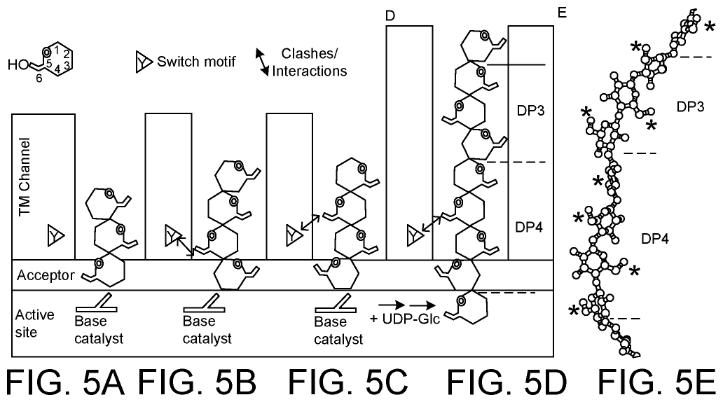

Synthesizing a (1,3:1,4)-β-glucan necessitates acceptor activation either at its C3 or C4 position (FIG. 5). The profound similarity of barley CslF6's catalytic domain with plant and bacterial CesAs suggests the presence of a single base catalyst only, i.e., Asp634 of the TED motif. Thus, introducing a (1,3)-β-linkage into an otherwise (1,4)-β-linked glucan requires repositioning the acceptor sugar (its C3 or C4 hydroxyl group) by about 2 Å relative to Asp634.

A sequence comparison of cellulose and (1,3:1,4)-β-glucan synthesizing enzymes provides insights into how (1,3)-β-linkages may be introduced. The identified switch motif lining the secretion channel is a defining sequence element of CslF6 enzymes. It is postulated that it recognizes specific features of the translocating glucan (FIG. 5).

In the (1,4)-β-glucan cellulose, neighboring glucosyl units are rotated by about 180 degrees relative to each other. This alternating orientation is established at the active site following chain elongation and positions the C6 hydroxyl groups of consecutive glucosyl units on opposing sides of the ribbon-shaped polymer (FIG. 5). A similar alternating orientation likely occurs between adjacent (1,4)-linked glucosyl residues in a (1,3:1,4)-β-glucan. Accordingly, register-dependent interactions of the switch motif with the nascent glucan could introduce the characteristic (1,3)-β-linkages by repositioning the acceptor sugar at the active site.

Proposed herein is that interactions of Tyr787 with the C6 hydroxyl of the polymer's penultimate glucosyl unit (counting from the non-reducing end) reposition the acceptor to favor glucosyl transfer to its C3 hydroxyl group, instead of its C4 hydroxyl. Applying this rule generates a glucan with only DP3 units upon lichenase digestion (FIG. 5). For stereochemical reasons, the C6 hydroxyl group of a (1,4)-β-linked glucosyl unit following a (1,3)-β-linked unit likely points to the same side of the polymer as the preceding sugar (FIG. 5E).

It is assumed that the acceptor can also be repositioned through interactions of the switch motif's Tyr787 with a C6 hydroxyl in the third position. This is supported (1) by its location relative to the nascent glucan (FIGS. 2 and 3C) and (2) by the orientation of the corresponding His residue in plant CesA structures (22, 27). Applying these ‘switch motif rules’ generates the characteristic DP3 and DP4 fragments upon lichenase hydrolysis (FIG. 5). Species-dependent differences in DP3:DP4 ratios may then reflect varying probabilities of acceptor repositioning due to interactions with the glucan's second or third glucosyl unit. This model agrees with previous results suggesting that residues located within CslF6's TM region indeed influence the DP3:DP4 ratios (34). Confirming this model will require high-resolution insights into CslF6-glucan translocation intermediates at different registers.

Hyaluronan synthase is another membrane-embedded polysaccharide synthase that creates (1,4)- and (1,3)-β-glycosidic linkages (38). In this case, however, linkage formation is dictated by the accepting glycosyl unit, which is either glucuronic acid or N-acetyl glucosamine. Failure to convert trimeric poplar CesA8 to a (1,3:1,4)-β-glucan synthase by introducing the barley CslF6 switch motif suggests that additional components are important to introduce (1,3)-β linkages. The monomeric functional unit of CslF6, with its altered position of TM helix 7, creates a different, slightly wider TM channel. The TM architecture of (1,3:1,4)-β-glucan synthase likely evolved to accommodate the nonlinearity of the synthesized (1,3:1,4)-β-glucan, in contrast to the linearity of cellulose synthesized by bona-fide CesAs.

The biochemical and structural analyses failed to reveal homo-oligomerization of CslF6 in vitro. Because (1,3:1,4)-β-glucans do not form fiber-like structures comparable to cellulose, the polymeric products released from monomeric or oligomeric forms of CslF6 may not differ substantially in their material properties. Of note, CslD, which is implicated in polarized cell wall cellulose deposition, has been shown to oligomerize in vitro, perhaps similar to bona-fide CesAs (37, 39). Because CslD produces cellulose, oligomerization may be necessary to spatio-temporally orchestrate glucan secretion to form fibrillar cellulosic materials (40). Thus, it is concluded that CslF6 preferentially functions as a monomer, although oligomerization in vivo mediated by other factors or oxidative processes cannot be excluded. The ability to modulate the structure of the CslF6-synthesized glucan through protein engineering enables the generation of genetic variants of barley and other cereal grains for human health and nutrition.

### BIBLIOGRAPHY

1. K. Keegstra, Plant cell walls. Plant Physiol 154, 483-486 (2010).
2. R. G. Staudte, J. R. Woodward, G. B. Fincher, B. A. Stone, Water soluble (1->3) (1->4)-b-D-glucans from barley (Hordeum vulgare) endosperm. Carbohydr Polym 3, 299-312 (1983).
3. G. S. Buliga, D. A. Brant, G. B. Fincher, The Sequence Statistics and Solution Conformation of a Barley (1-]3, 1-]4)-Beta-D-Glucan. Carbohydrate Res 157, 139-156 (1986).
4. C. S. Brennan, L. J. Cleary, The potential use of cereal (1->3,1->4)-beta-D-glucans as functional food ingredients. J Cereal Sci 42, 1-13 (2005).
5. H. Collins et al., Variability in fine structures of noncellulosic cell wall polysaccharides from cereal grains: potential importance in human health and nutrition. Ceral Chem 87, 272-282 (2010).
6. A. Lazaridou, C. G. Biliaderis, Molecular aspects of cereal beta-glucan functionality: Physical properties, technological applications and physiological effects. J Cereal Sci 46, 101-118 (2007).
7. M. A. Anderson, B. A. Stone, A new substrate for investigating the specificity of beta-glucan hydrolases. FEBS Lett 52, 202-207 (1975).
8. V. Lombard, H. G. Ramulu, E. Drula, P. M. Coutinho, B. Henrissat, The carbohydrate-active enzymes database (CAZy) in 2013. Nucleic Acids Res 42, D490-D495 (2014).
9. J. L. Morgan et al., Observing cellulose biosynthesis and membrane translocation in crystallo. Nature 531, 329-334 (2016).
10. L. L. Lairson, B. Henrissat, G. J. Davies, S. G. Withers, Glycosyltransferases: structures, functions, and mechanisms. Annu Rev Biochem 77, 521-555 (2008).
11. A. Little et al., Revised Phylogeny of the Cellulose Synthase Gene Superfamily: Insights into Cell Wall Evolution. Plant Physiol 177, 1124-1141 (2018).
12. R. A. Burton et al., Cellulose synthase-like CslF genes mediate the synthesis of cell wall (1,3;1,4)-beta-D-glucans. Science 311, 1940-1942 (2006).
13. M. S. Doblin et al., A barley cellulose synthase-like CSLH gene mediates (1,3;1,4)-beta-D-glucan synthesis in transgenic Arabidopsis. Proc Natl Acad Sci USA 106, 5996-6001 (2009).
14. S. P. Hazen, J. S. Scott-Craig, J. D. Walton, Cellulose synthase-like genes of rice. Plant Physiol 128, 336-340 (2002).
15. J. G. Schwerdt et al., Evolutionary Dynamics of the Cellulose Synthase Gene Superfamily in Grasses. Plant physiology 168, 968-983 (2015).
16. Y. Yin, M. A. Johns, H. Cao, M. Rupani, A survey of plant and algal genomes and transcriptomes reveals new insights into the evolution and function of the cellulose synthase superfamily. BMC Genomics 15, 260 (2014).
17. C. M. Kim et al., OsCSLD1, a cellulose synthase-like D1 gene, is required for root hair morphogenesis in rice. Plant Physiol 143, 1220-1230 (2007).
18. A. J. Bernal et al., Functional Analysis of the Cellulose Synthase-Like Genes CSLD1, CSLD2, and CSLD4 in Tip-Growing Arabidopsis Cells. Plant Physiol 148, 1238-1253 (2008).
19. S. J. Kim, S. Zemelis, K. Keegstra, F. Brandizzi, The cytoplasmic localization of the catalytic site of CSLF6 supports a channeling model for the biosynthesis of mixed-linkage glucan. Plant J 81, 537-547 (2015).
20. R. A. Burton, M. J. Gidley, G. B. Fincher, Heterogeneity in the chemistry, structure and function of plant cell walls. Nat Chem Biol 6, 724-732 (2010).
21. G. Dimitroff et al., (1,3;1,4)-beta-Glucan Biosynthesis by the CSLF6 Enzyme: Position and Flexibility of Catalytic Residues Influence Product Fine Structure. Biochemistry 55, 2054-2061 (2016).
22. P. Purushotham, R. Ho, J. Zimmer, Architecture of a catalytically active homotrimeric plant cellulose synthase complex. Science 369, 1089-1094 (2020).
23. P. Purushotham et al., A single heterologously expressed plant cellulose synthase isoform is sufficient for cellulose microfibril formation in vitro. Proc Natl Acad Sci USA 113, 11360-11365 (2016).
24. L.-l. Fu et al., High-level secretion of a chimeric thermostable lichenase from Bacillus subtilis by screening of site-mutated signal peptides with structural alterations. Curr Microbiol 56, 287-292 (2008).
25. A. Punjani, J. L. Rubinstein, D. J. Fleet, M. A. Brubaker, cryoSPARC: algorithms for rapid unsupervised cryo-EM structure determination. Nat Methods 14, 290-296 (2017).
26. S. H. W. Scheres, RELION: Implementation of a Bayesian approach to cryo-EM structure determination. J Struct Biol 180, 519-530 (2012).
27. X. Zhang et al., Structural insights into homotrimeric assembly of cellulose synthase CesA7 from Gossypium hirsutum. Plant Biotechnol J, (2021).
28. J. Jumper et al., Highly accurate protein structure prediction with AlphaFold. Nature 596, 583-589 (2021).
29. K. Tunyasuvunakool et al., Highly accurate protein structure prediction for the human proteome. Nature 596, 590-+ (2021).
30. J. Morgan, J. Strumillo, J. Zimmer, Crystallographic snapshot of cellulose synthesis and membrane translocation. Nature 493, 181-186 (2013).
31. C. E. Vergara, N. C. Carpita, Beta-D-glycan synthases and the CesA gene family: lessons to be learned from the mixed-linkage (1->3), (1->4) beta-D-glucan synthase. Plant Mol Biol 47, 145-160 (2001).
32. P. S. Rushton et al., Rice Cellulose SynthaseA8 Plant-Conserved Region Is a Coiled-Coil at the Catalytic Core Entrance. Plant Physiol 173, 482-494 (2017).
33. T. R. Scavuzzo-Duggan et al., Cellulose synthase ‘class specific regions’ are intrinsically disordered and functionally undifferentiated. J Integr Plant Biol 60, 481-497 (2018).
34. S. A. Jobling, Membrane pore architecture of the CslF6 protein controls (1-3,1-4)-beta-glucan structure. Sci Adv 1, e1500069 (2015).
35. M. Pauly et al., Hemicellulose biosynthesis. Planta 238, 627-642 (2013).
36. A. H. Liepman, D. M. Cavalier, The CELLULOSE SYNTHASE-LIKE A and CELLULOSE SYNTHASE-LIKE C families: recent advances and future perspectives. Front Plant Sci 3, (2012).
37. J. Yang et al., Cellulose synthase-like D (CSLD) 3 protein is a beta-1,4-glucan synthase Plant Cell 32, 1749-1767 (2020).
38. F. P. Maloney et al., Structure, substrate recognition and initiation of hyaluronan synthase. Nature 604, 195-+ (2022).
39. J. Yang et al., Biochemical and Genetic Analysis Identify CSLD3 as a beta-1,4-Glucan Synthase That Functions during Plant Cell Wall Synthesis. The Plant cell 32, 1749-1767 (2020).
40. H. Yang, J. D. Kubicki, A density functional theory study on the shape of the primary cellulose microfibril in plants: effects of C6 exocyclic group conformation and H-bonding. Cellulose 27, 2389 (2020).
41. F. A. Pettolino, C. Walsh, G. B. Fincher, A. Bacic, Determining the polysaccharide composition of plant cell walls. Nat Protoc 7, 1590-1607 (2012).
42. P. Emsley, K. Cowtan, Coot: model-building tools for molecular graphics. Acta Crystallogr D Biol Crystallogr 60, 2126-2132 (2004).
43. P. V. Afonine et al., Towards automated crystallographic structure refinement with phenix.refine. Acta Crystallogr D Biol Crystallogr 68, 352-367 (2012).
44. R. C. Edgar, MUSCLE: multiple sequence alignment with high accuracy and high throughput. Nucl Acid Res 32, 1792-1797 (2004).
45. A. M. Waterhouse, J. B. Procter, D. M. Martin, M. Clamp, G. J. Barton, Jalview Version 2—a multiple sequence alignment editor and analysis workbench. Bioinform 25, 1189-1191 (2009).

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A cereal plant or a part thereof, wherein the plant or part thereof has an altered (1,3;1,4)-β-glucan content as compared to a wild-type cereal plant or part thereof, wherein said plant or part thereof carries one or more mutations in the CslF6 gene, wherein said mutated CslF6 gene encodes a mutant CslF6 polypeptide, wherein said mutant CslF6 comprises at least one substitution, addition or deletion of an amino acid in a switch motif of CslF6, wherein the switch motif comprises SEQ ID NO:14.

2. The plant or plant part thereof of claim 1, wherein one or more amino acids are substituted in the switch motif.

3. The plant or plant part thereof of claim 1, wherein the tyrosine of the switch motif is substituted with a histidine.

4. The plant or plant part thereof of claim 1, wherein the substitution, addition or deletion results in a reduction of (1,3)-β-linkage formation as compared to wild type.

5. The plant or plant part thereof of claim 1, wherein the altered plant (1,3;1,4)-β-glucan content results in a ratio DP3:DP4 of 1:2.5 to 1:32.

6. The plant or plant part thereof of claim 1, wherein the substitution, addition or deletion results in increase of (1,3)-β-linkage formation as compared to wild type.

7. A composition comprising the plant or a part thereof of claim 1.

8. A food or drink product comprising the plant or a part thereof of claim 1.

9. The food or drink product of claim 8, wherein the food or drink product is selected from a malt beverage, a flour, a syrup, a malt, a beer, a bagel, a biscuit, a bread, a bun, a croissant a dumpling, an English muffin, a muffin, a pita bread, a quick bread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut an egg roll, an extruded snack, a fruit and grain bar, a microwaveable snack product a nutritional bar, a pancake, a par-baked bakery product, a pretzel, a pudding, a granola-based product, a snack chip, a snack food, a snack mix, a waffle, a pizza crust, animal food or pet food.

10. A food additive, bulking agent, dietary fiber, texturizing agent, preservative or probiotic agent made from the plant or part there of claim 1.

11. A method to treat or improve the health of an animal, comprising administering the plant or a part thereof of claim 1 to the animal in need thereof so as to treat or improve said animal's health.

12. The method of claim 11, wherein metabolic health, bowel health or cardiovascular health is improved or the severity of metabolic, bowel or cardiovascular disease is decreased in said animal.

13. The method of claim 11, wherein the animal is a mammal.

14. The method of claim 13, wherein the mammal is a human.


SEQUENCE LISTING (66 sequences)

SEQ ID NO: 1 - DNA, 78 bp, synthetic construct
  source 1..78: mol_type = other DNA
acaataagcggccgcatgcaccatcaccatcaccatcaccatcaccatcaccatatgcaccatcaccatcaccatcac

SEQ ID NO: 2 - DNA, 40 bp, synthetic construct
  source 1..40: mol_type = other DNA
tataacgaaagctttcaatggagccagccatagagccctg

SEQ ID NO: 3 - DNA, 39 bp, synthetic construct
  source 1..39: mol_type = other DNA
gagatcggctgggtgtacggcaccgtcacggaggacgtg

SEQ ID NO: 4 - DNA, 39 bp, synthetic construct
  source 1..39: mol_type = other DNA
cacgtcctccgtgacggtgccgtacacccagccgatctc

SEQ ID NO: 5 - DNA, 45 bp, synthetic construct
  source 1..45: mol_type = other DNA
tgttctgctcacctggctgcagtgtgccaggtgctgaccaaggtg

SEQ ID NO: 6 - DNA, 45 bp, synthetic construct
  source 1..45: mol_type = other DNA
tgcagccaggtgagcagaacaggatgctgtcatccagaactggcc

SEQ ID NO: 7 - DNA, 45 bp, synthetic construct
  source 1..45: mol_type = other DNA
atcggtggcgtgagcgctcacctcgccgccgtctgccaggtgctg

SEQ ID NO: 8 - DNA, 45 bp, synthetic construct
  source 1..45: mol_type = other DNA
agcgctcacgccaccgatcacccagaactggccgttcctgaacca

SEQ ID NO: 9 - DNA, 45 bp, synthetic construct
  source 1..45: mol_type = other DNA
gtgtccgcttacctgttcgctgtgttccagggattcttgaaaatg

SEQ ID NO: 10 - DNA, 45 bp, synthetic construct
  source 1..45: mol_type = other DNA
agcgaacaggtaagcggacacgcctccgatcacccagaactgttc

SEQ ID NO: 11 - DNA, 45 bp, synthetic construct
  source 1..45: mol_type = other DNA
accgcttcctgcagcgcttacctctttgccgtcttccagggattc

SEQ ID NO: 12 - DNA, 45 bp, synthetic construct
  source 1..45: mol_type = other DNA
agcgctgcaggaagcggtcatccagaactgttcattacgccacaa

SEQ ID NO: 13 - protein, 8 aa, Hordeum vulgare
VIGGVSAH

SEQ ID NO: 14 - protein, 8 aa, Hordeum vulgare
MTASCSAY

SEQ ID NO: 15 - protein, 947 aa, Hordeum vulgare
MAPAVAGGGRVRSNEPVAAAAAAPAASGKPCVCGFQVCACTGSAAVASAASSLDMDIVAMGQIGAVNDESWVGVELGEDGETDESGAAVDDRPVFRTEKIKGVLLHPYRVLIFVRLIAFTLFVIWRISHKNPDAMWLWVTSICGEFWFGFSWLLDQLPKLNPINRVPDLAVLRQRFDRPDGTSTLPGLDIFVTTADPIKEPILSTANSVLSILAADYPVDRNTCYVSDDSGMLLTYEALAESSKFATLWVPFCRKHGIEPRGPESYFELKSHPYMGRAQDEFVNDRRRVRKEYDEFKARINSLEHDIKQRNDGYNAAIAHSQGVPRPTWMADGTQWEGTWVDASENHRRGDHAGIVLVLLNHPSHRRQTGPPASADNPLDLSGVDVRLPMLVYVSREKRPGHDHQKKAGAMNALTRASALLSNSPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPGLEMTTAKAKAAPVPAKGKHGFLPLPKKTYGKSDAFVDTIPRASHPSPYTAAAEGIVADEATIVEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVLYINIPHMHTSGGKHTTVHGHHGKKLVDTGLYGWLH

SEQ ID NO: 16 - DNA, 5100 bp, Hordeum vulgare
  source 1..5100: mol_type = other DNA
atggcgccagcggtggccggagggggccgcgtgcggagcaatgagccggttgctgctgctgccgccgcgccggcggccagcggcaagccctgcgtgtgcggcttccaggtttgcgcctgcacggggtcggccgcggtggcctccgccgcctcgtcgctggacatggacatcgtagccatggggcagatcggcgccgtcaacgacgagagctgggtgggcgtggagctcggcgaagatggcgagaccgacgaaagcggtgccgccgttgacgaccgccccgtattccgcaccgagaagatcaagggtgtcctcctccacccctaccggtacgtcctgctcccacaactaaacagaaactccctatatctgcgtcacactcaacaattaatccaactaagtctctctactactatagtatttatttttactctctatctgcacaacaagcgctactacaattaacccaacaagcaccacgccaggttgacagtcaggataatttgatcttgaccggagtaagtactagtactaggtcggtgttaatcagagtaattattgcactagttaattaaaatttgagtaatccgagacaggtgcacgttagggccgggccaatgatcgctcgaatccacccaaaatagcgcgtcccggtgtgggctgtcggctcggtgcttcttccttccattttactagtcgcagtcactgcagcttgggcccacgggaggggacgttagccgttgggcctgccaggcatgtgggccccggtggccaccctggcggctcaggtgggccccggtggccaccctggcggctcataaatccttgctactttggagctgtaatggacgctctgcaatagcaataggaatccgaggtgaaacgacgacagtgggcatggcatggcttgcatgtgaatccaagccacatcattaaaagcatcctccctgggcacgtcgcggtgagaaagttggataaacttttgggggttcggacaagatgagaaaaagcaagtaacatggcccttttttggcaccgaagaaatcctatgtacggcgagttttttctgcatctagttatgggtagatgtacgttagtttttgttgagcgttttatgtgctacatatatggagaaaaagagaaaaatattatcatgtcatgtcatgccatgagaagaaaaagaagtgttgccaccgctcgaatgcctcttttttctttcggaaggatgcgtgagtcatgttggcaccgagaaaagccatattaaagtggcagagttacaactcagaataaatgcgggtgttacaaaaacactaggatatgtgaagggcactcggcacaacactttaagactacacaattgaaaaagtgtactctctgtatctaaataattataattgaaaagaattaatctatatatgaaagtagtaatgattagcggtagaaggttccaacgacttttttggcgccaatagcaagaagaaagaaaaagaaaaaaatctttactgtacagtaacgagaaaagaggcccattaatagagcaacgaatccagcggcaccacctctggcggtcacgtccatgccctcgcacgacggatgaggcccggggggtcctactgacagccgaagcatgtcggtgctcaaacacggcgccgtttgctgccaagtgtgccagctcgcactcattgacttgccagctctctccttggttgtcaatgagaacatgatgccttttggcatttgcaaacttattaaaactagctgtcgtccgatagggaaaagaaaagaaaagaaaagaataagaaaaaaaggacaaagagaaaagatgaacatggcgcatgttccctccaataattgcaggcaccaacactgggtcgattaatccaacaacaatattttactataccagacgagagtacagtagtcgggtgatgatggactgtaactgactgagtatgaatgactgtaatgcagggtgctgattttcgttcgtctgatcgccttcacgctgttcgtgatctggcgtatctcccacaagaacccagacgcgatgtggctgtgggtgacatccatctgcggcgagttctggttcggtttctcgtggctgctggatcagctgcccaagctgaaccccatcaaccgcgtgccggacctggcggtgctgcggcagcgcttcgaccgccccgacggcacctccacgctcccggggctggacatcttcgtcaccacggccgaccccatcaaggagcccatcctctccaccgccaactcggtgctctccatcctggccgccgactaccccgtggaccgcaacacatgctacgtctccgacgacagtggcatgctgctcacctacgaggccctggcagagtcctccaagttcgccacgctctgggtgcccttctgccgcaagcacgggatcgagcccaggggtccggagagctacttcgagctcaagtcacacccttacatggggagagcccaggacgagttcgtcaacgaccgccgccgcgttcgcaaggagtacgacgagttcaaggccaggatcaacagcctggagcatgacatcaagcagcgcaacgacgggtacaacgccgccattgcccacagccaaggcgtgccccggcccacctggatggcggacggcacccagtgggagggcacatgggtcgacgcctccgagaaccaccgcaggggcgaccacgccggcatcgtactggtcagtatccatccatctttctgctgcttatattactcttaggttactcttatcgtctctttcctataccgtacatgcatgcatgctgctattcttggaatcgtggttggttactactccaccatgcaaaaataacaagaagaggaatcttggttagttagggcctcgttgttatattagtggccatctgatgtgatgcctgccggctgtgcccatccatatccatggaagatttcgacagaatcgacgtggtgatagttgagagtgcaaccaccacccagagccagccaagcacatgcatgcttctcttctcgtctcgtcgtgtggccagcagcgcattcatgctattgctgtgacgagggaggaatggtggttggggtggtcctttccccccgacagcactacagcctccactttatgacccatttaattcatcggccctgctttgttgtaaccgccttctcacctcaatcaatcattcattcattcataagtttactcactctttgttactactcgaaccactaatcaggaaggagtaggagtaatgcagatttactattaacagttaaaggagtaaaaagaaggaagcacaattacagaaccttgtttttttttactactgtacgtaaggtgtaagaatggagtgctgacagagaatggatgcaggtgctgctgaaccacccgagccaccgccggcagacgggcccgccggcgagcgctgacaacccactggacttgagcggcgtggatgtgcgtctccccatgctggtgtacgtgtcccgtgagaagcgccccgggcacgaccaccagaagaaggccggtgccatgaacgcgcttacccgcgcctcggcgctgctctccaactcccccttcatcctcaacctcgactgcgatcattacatcaacaactcccaggcccttcgcgccggcatctgcttcatggtgggacgggacagcgacacggttgccttcgtccagttcccgcagcgcttcgagggcgtcgaccccaccgacctctacgccaaccacaaccgcatcttcttcgacggcaccctccgtgccctggacggcatgcagggccccatctacgtcggcactgggtgtctcttccgccgcatcaccgtctacggcttcgacccgccgaggatcaacgtcggcggtccctgcttccccaggctcgccgggctcttcgccaagaccaagtacgagaagcccgggctcgagatgaccacggccaaggccaaggccgcgcccgtgcccgccaagggtaagcacggcttcttgccactgcccaagaagacgtacggcaagtcggacgccttcgtggacaccatcccgcgcgcgtcgcacccgtcgccctacaccgcggcggctgaggggatcgtggccgacgaggcgaccatcgtcgaggcggtgaacgtgacggccgccgcgttcgagaagaagaccggctggggcaaagagatcggctgggtgtacgacaccgtcacggaggacgtggtcaccggctaccggatgcatatcaaggggtggcggtcacgctactgctccatctacccacacgccttcatcggcaccgcccccatcaacctcacggagaggctcttccaggtgctccgctggtccacgggatccctcgagatcttcttctccaagaacaacccgctcttcggcagcacatacctccacccgctgcagcgcgtcgcctacatcaacatcaccacttaccccttcaccgccatcttcctcatcttctacaccaccgtgccggcgctatccttcgtcaccggccacttcatcgtgcagcgcccgaccaccatgttctacgtctacctgggcatcgtgctatccacgctgctcgtcatcgccgtgctggaggtcaagtgggccggggtcacagtcttcgagtggttcaggaacggccagttctggatgacagcaagttgctccgcctacctcgccgccgtctgccaggtgctgaccaaggtgatattccggcgggacatctccttcaagctcacatccaagctaccctcgggagacgagaagaaggacccctacgccgacctctacgtggtgcgctggacgccgctcatgatcacacccatcatcatcatcttcgtcaacatcatcggatccgccgtggccttcgccaaggttctcgacggcgagtggacgcactggctcaaggtcgccggcggcgtcttcttcaacttctgggtgctcttccacctctaccccttcgccaagggcatcctggggaagcacggaaagacgccagtcgtggtgctcgtctggtgggcattcaccttcgtcatcaccgccgtgctctacatcaacatcccccacatgcatacctcgggaggcaagcacacaacggtgca

SEQ ID NO: 17 - protein, 852 aa, Oryza sativa
MVIGCCRVLIFVRLIAFTLFVIWRIEHKNPDAMWLWVTSIAGEFWFGFSWLLDQLPKLNPINRVPDLAVLRRRFDHADGTSSLPGLDIFVTTADPIKEPILSTANSILSILAADYPVDRNTCYLSDDSGMLLTYEAMAEAAKFATLWVPFCRKHAIEPRGPESYFELKSHPYMGRAQEEFVNDRRRVRKEYDDFKARINGLEHDIKQRSDSYNAAAGVKDGEPRATWMADGSQWEGTWIEQSENHRKGDHAGIVLVLLNHPSHARQLGPPASADNPLDFSGVDVRLPMLVYVAREKRPGCNHQKKAGAMNALTRASAVLSNSPFILNLDCDHYINNSQALRAGICFMLGRDSDTVAFVQFPQRFEGVDPTDLYANHNRIFFDGTLRALDGLQGPIYVGTGCLFRRITLYGFEPPRINVGGPCFPRLGGMFAKNRYQKPGFEMTKPGAKPVAPPPAATVAKGKHGFLPMPKKAYGKSDAFADTIPRASHPSPYAAEAAVAADEAAIAEAVMVTAAAYEKKTGWGSDIGWVYGTVTEDVVTGYRMHIKGWRSRYCSIYPHAFIGTAPINLTERLFQVLRWSTGSLEIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVLQVVTKVVFRRDISFKLTSKLPAGDEKKDPYADLYVVRWTWLMITPIIIILVNIIGSAVAFAKVLDGEWTHWLKVAGGVFFNFWVLFHLYPFAKGILGKHGKTPVVVLVWWAFTFVITAVLYINIPHIHGPGRHGAASPSHGHHSAHGTKKYDFTYAWP

SEQ ID NO: 18 - protein, 934 aa, Eragrostis curvula
MAPGVGDGRRNNGEVVPAGGGRCVCGFQVCACAGAAAVASAASSADMDKMALAATATEGQIGAVNDESWVAVDLSDDLSGDGADAGVALEDRPVFRTEKIKGILLHPYRVLIFVRLIAFSLFVVWRISHKNPDAISGSVSPGCWINSKTEPDQPRPGPGGAPAALRPGRRHIPPAGLDIFVTTADPFKEPILSTANSILSILAADYPVEKNTCYLSDDSGMLLTYEAMAEAAKFATVWVPFCRKHGIEPRGPESYFELKSHPYMGRAQEDFVNDRRRVRKEYDEFKARINGLEHDIKQRSDGYNANVKDGEPRATWMSDGTQWQGTWVEPSENHRKGDHAGIVLVLLNHPSHTRQLGPPASADNPLDFSLVDVRLPMLVYVAREKRPGHNHQKKAGAMNALTRASAVLSNSPFILNLDCDHYINNSQALRAGICFMLGRDSDTVAFVQFPQRFEGVDPTDLYANHNRIFFDGTLRALDGMQGPIYVGTGCLFRRTTLYGFDPPRINVGGPCFPMLGGMFAKTKYEKPGLELTTKAATIAKGKHGFLPMPKKSYGKSDAFVDTIPKASHPSPYSAAEAAVVADEAAIAEAVAVCTAAYEKKTGWGSSIGWVYGTVTEDVVTGYRMHIKGWRSRYCSIYPHAFIGTAPINLTERLYQVLRWSTGSLEIFFSKNNPLFGSTFLHPLQRVAYINITTYPFTAIFLIFYTTVPALSFVTGHFIVQRPTTMFYVYLLIVLGTLLILAVLEVKWAGVTVFEWFRNGQFWMTASCSAYLAAVLQVLFKVVFRRDISFKLTSKQPAGDEKKDPYADLYVVRWTWLMVTPIIIILVNIIGSAVAFAKVLDGEWTHWLKVAGGVFFNFWVLFHLYPFAKGLLGKHGKTPVVVLVWWAFTFVITAVLYINIPHFHASGGGHHAAKHGGHGAHHGTKHLDHVFFGWP

SEQ ID NO: 19 - protein, 712 aa, Zea mays
MLLTYEAMAEAAKFATVWVPFCRKHGIEPRGPESYFELKSHPYMGRSQEDFVNDRRRVRRDYDEFKARINGLENDIRQRSDAYNAARGLKDGEPRATWMADGTQWEGTWVEPSENHRKGDHAGIVLVLLNHPSHSRQLGPPASADNPLDLSMVDVRLPMLVYVSREKRPGHNHQKKAGAMNALTRCSAVLSNSPFILNLDCDHYINNSQALRAGICFMLGRDSDTVAFVQFPQRFEGVDPTDLYANHNRIFFDGTLRALDGMQGPIYVGTGCLFRRITLYGFDPPRINVGGPCFPSLGGMFAKTKYEKPGLELTTKAAVAKGKHGFLPMPKKSYGKSDAFADTIPMASHPSPFAAAAAVVAEEATIAEAVAVCAAAYEKKTGWGSDIGWVYGTVTEDVVTGYRMHIKGWRSRYCSIYPHAFIGTAPINLTERLFQVLRWSTGSLEIFFSRNNPLFGSTFLHPLQRVAYINITTYPFTAIFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSKQPAGDEKKDPYADLYVVRWTWLMVTPIIIILVNIIGSAVAFAKVLDGEWTHWLKVAGGVFFNFWVLFHLYPFAKGILGRHGKTPVVVLVWWAFTFVITAVLYINIPHIHGPGGKHGGAIGKHGAAHHGKKFDGYYLWP

SEQ ID NO: 20 - protein, 961 aa, Sorghum bicolor
MAPGGGDGRRNGEGQQQANGNNNNNNSNAKAKHGCVCGFPVCACAGAAAVASAASSADMDRVAAAQTEGQIGAVNDESWIAVDLSDDLSGDGGGADPGVAIEDRPVFRTEKIKGILLHPYRVLIFVRLIAFTLFVIWRISHRNPDAMWLWVTSIAGEFWFGFSWLLDQLPKLNPINRVPDLAVLRQRFDRADGTSRLPGLDIFVTTADPFKEPILSTANSILSILAADYPVERNTCYLSDDSGMLLTYEAMAEAAKFATVWVPFCRKHGIEPRGPESYFELKSHPYMGRSQEDFVNDRRRVRKEYDEFKARINGLEHDIKQRSDAFNAARGLKDGEPRATWMADGNQWEGTWVEPSENHRKGDHAGIVYVLLNHPSHSRQLGPPASADNPLDFSMVDVRLPMLVYVSREKRPGFNHEKKAGAMNALTRCSAVISNSPFILNLDCDHYINNSQALRAGICFMLGRDSDTVAFVQFPQRFEGVDPTDLYANHNRIFFDGTLRALDGMQGPIYVGTGCMFRRITLYGFDPPRINVGGPCFPSLGGMFAKTKYEKPGLELTTKAAVAKGKHGFLPLPKKSYGKSDAFVDTIPRASHPSPFLSADEAAAIVADEAMITEAVEVCTAAYEKKTGWGSDIGWVYGTVTEDVVTGYRMHIKGWRSRYCSIYPHAFIGTAPINLTERLYQVLRWSTGSLEIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSKQPAGDEKKDPYADLYVVRWTWLMVTPIIIILVNIIGSAVAFAKVLDGEWTHWLKVAGGVFFNFWVLFHLYPFAKGLLGRHGKTPVVVLVWWAFTFVITAVLYINIPHIHGPGGKHGGAIGKHGAAHHGKKFDLDNLSYNWP

SEQ ID NO: 21 - protein, 952 aa, Miscanthus lutarioriparius
MAPGGGDGRRNGEAATGQQANGGNNAKAKHGCVCGFPVCACAGAAAVASAASSADMDRVAVAATEGQIGAVNDESWIAVDLSDDLSGDGADPGVALEDRPVFRTEKIKGILLHPYRVLIFVRLIAFTLFVIWRISHRNPDALWLWVTSIAGEFWFGFSWLLDQLPKLNPINRVPDLAVLRQRFDRADGTSLLPGLDIFVTTADPFKEPILSTANSILSILAADYPVERNTCYLSDDSGMLLTYEAIAEAAKFATVWVPFCRKHGIEPRGPESYFELKSHPYIGRSQEDFVNDRRRVRKEYDEFKARINGLEHDIKQRSDAYNAARGLKDGEPRATCMADGNQWEGTWVEPSENHRKGDHAGIVYVLLNHPSHSRQLGPPASADNPLDFSMVDVRLPMLVYVSREKRPGFNHEKKAGAMNALTRCSAVISNSPFILNLDCDHYINNSQALRAGICFMLGRDSDTVAFVQFPQRFEGVDPTDLYANHNRIFFDGTLRALDGMQGPIYVGTGCMFRRITLYGFDPPRINVGGPCFPSLGGMFTKTKYEKPGLELTTKAAVAKGKHGFLPLPKKSYGKSDAFVDTIPRASHPSPFADADEAAAIVADEATITESVAVCTAAYEKKTGWGSDIGWVYGTVTEDVVTGYRMHIKGWRSRYCSIYPHAFIGTAPINLTERLYQVLRWSTGSLEIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSKQPAGDEKKDPYADLYVVRWTWLMVTPIIIILVNIIGSAVAFAKVLDGEWTHWLKVAGGVFFNFWVLFHLYPFAKGILGKHGKTPVVVLVWWAFTFVITAVLYINIPHIHGPGGKHGGAIGKHGAAHHGKRYELYGWP

SEQ ID NO: 22 - protein, 956 aa, Setaria italica
MAPGGGDGRRNNGEQANGNNRHGCVCGFPVCACAGAAAVASAASSADMDRVVAVAATEGQIGAVNDESWVAVDLSDDLSGDGGDDGVAIEDRPVFRTEKIKGVLLHPYRVLIFVRLIAFTLFVIWRISHRNPDAQWLWVTSIAGEFWFGFSWLLDQLPKLNPINRVPDLAVLRQRFDRADGTSRLPGLDIFVTTADPFKEPILSTANSILSILAADYPVEKNTCYLSDDSGMLLTYEAMVEAAKFATVWVPFCRKHGIEPRGPESYFELKSHPYMGRSQEDFVNDRRRVRKEYDEFKARINGLEHDIKQRSDAYNAARGLKDGEPRATWMADGNQWEGTWVEPSENHRKGDHTGIVLVLVNHPSHGRQFGPPASADNPLDFSMVDVRLPMLVYVSREKRPGFNHEKKAGAMNALTRCSAVLTNSPFILNLDCDHYINNSQALRAGICFMLGRDSDTVAFVQFPQRFEGVDPTDLYANHNRIFFDGTLRALDGMQGPIYVGTGCLFRRVTLYGFDPPRINVGGQCFPSLGGMFAKTKYEKPGLEMSTAKGAATAVVAKGKHGFLPLPKKSYGKSEAFVDSIPRASHPSPFANATGDAGVLTDEATISEAVAVTTAAYEKKTGWGSNIGWVYGTVTEDVVTGYRMHIKGWRSRYCSIYPHAFIGTAPINLTERLYQVLRWSTGSLEIFFSKNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVVVKVVFRRDISFKLTSKQPAGDEKKDPYADLYVVRWTWLMVMPIIIILVNIIGSAVAFAKVLDGEWTHWLKVAGGVFFNFWVLFHLYPFAKGILGKHGKTPVVVLVWWAFTFVITAVLYINIPHIHGPGGKHGHGGALGKHAHGHHAGSKFGYSEVYGWP

SEQ ID NO: 23 - protein, 942 aa, Dichanthelium oligosanthes
MAPGGGDGRRNGEAQANGKHGCACGFPVCACAGAAAVASAASSADMDRVAVAATEGQIGAVNDESWVAVDLSDDLSGDGADDGVAIEDRPVFRTEKIKGILLHPYRVLIFVRLIAFTLFVIWRISHRNPDAQWLWVTSIAGEFWFGFSWLLDQLPKLNPINRVPDLAVLRQRFDRVDGTSRLPGLDIFVTTADPFKEPILSTANSILSILAADYPVERNTCYLSDDSGMLLTYEAMVEAAKFATVWVPFCRKHGIEPRGPESYFELKSHPYMGRSQEDFVNDRRRVRKEYDEFKARINGLEHDIKQRSDAYNAARGLKDGEPRATWMADGSQWEGTWVEPSENHRKGDHAGIVLMLVNHPSHTRQLGPPASADNPLDFSMVDVRLPMLVYVAREKRPGFNHEKKAGAMNALTRCSAVLTNSPFILNLDCDHYINNSQALRAGICFMLGRDSDTVAFVQFPQRFEGVDPTDLYANHNRIFFDGTLRALDGMQGPIYVGTGCLFRRITLYGFDPPRINVGGPCFPALGGMFAKTKYEKPGLEMTTKAAVAKGKHGFLPLPKKSYGKSDAFVDSIPRASHPSPYAEAEAVVATDEATITEAVAVTTAAYEKKTGWGSSIGWVYGTVTEDVVTGYRMHIKGWRSRYCSIYPHAFIGTAPINLTERLYQVLRWSTGSLEIFFSKNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSKQPSGDEKKDPYADLYVVRWTWLMVTPIIIILVNIIGSAVAFAKVLDGEWTHWLKVAGGVFFNFWVLFHLYPFAKGILGKHGKTPVVVLVWWAFTFVITAVLYINIPHIHGPGGGKHGGVLGKHAAHRGHHGGGEAYGWP

SEQ ID NO: 24 - protein, 946 aa, Panicum hallii
MAPGGGDGRRNGEQPQQASGGRHGCACGFPVCACAGAAAVASAASSADMDRAAVAATEGQIGAVNDESWVAVDLSDDLSGDGGDDGVAIEDRPVFRTEKIKGVLLHPYRVLIFVRLIAFTLFVIWRISHRNPDALWLWVTSIAGEFWFGFSWLLDQLPKLNPINRVPDLAVLRQRFDRADGTSRLPGLDIFVTTADPFKEPILSTANSILSILAADYPVEKNTCYLSDDSGMLLTYEAMVEAAKFATVWVPFCRKHGIEPRGPESYFELKSHPYMGRSQEDFVNDRRRVRKEYDEFKARINGLEHDIKQRSDAYNAARGLKDGEPRATWMADGSQWEGTWVEPSENHRKGDHAGIVLVLVNHPSHSRQLGPPASADNPLDFSMVDVRLPMLVYVSREKRPGFNHEKKAGAMNALTRCSAVLTNSPFILNLDCDHYINNSQALRAGICFMLGRDSDTVAFVQFPQRFEGVDPTDLYANHNRIFFDGTLRALDGMQGPIYVGTGCLFRRVTLYGFDPPRINVGGPCFPSLGGMFAKTKYEKPGLEMTTKAAVAKGKHGFLPLPKKAYGKSDAFVDSIPRASHPSPFADAAAVVADEATISEAVAVTTAAYEKKTGWGSNIGWVYGTVTEDVVTGYRMHIKGWRSRYCSIYPHAFIGTAPINLTERLYQVLRWSTGSLEIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVVVKVVFRRDISFKLTSKQPAGDEKKDPYADLYVVRWTWLMVTPIIIILVNIIGSAVAFAKVLDGEWTHWLKVAGGVFFNFWVLFHLYPFAKGILGKHGKTPVVVLVWWAFTFVITAVLYINIPHIHGPGGKHGHGGGMGRHAHHHAGSTHVTELYGWP

SEQ ID NO: 25 - protein, 843 aa, Panicum virgatum
MAWHGRVLIFVRLIAFTLFVIWRISHRNPDALWLWVTSIAGEFWFGFSWLLDQLPKLNPINRVPDLAVLRQRFDRADGTSRLPGLDIFVTTADPFKEPILSTANSVLSILAADYPVERNTCYLSDDSGMLLTYEAMAEAAKFATVWVPFCRKHGIEPRGPESYFELKSHPYMGRSQEDFVNDRRRVRKEYDEFKARINGLEHDIKQRSDAHNAARGLKDGQPRATWMADGTQWEGTWVEPAENHRKGDHAGIVQVLVNHPSHSRQLGPPASADNPLDFSMVDVRLPMLVYVSREKRPGFNHEKKAGAMNALTRCSAVLTNSPFILNLDCDHYINNSQALRAGICFMLGRDSDTVAFVQFPQRFEGVDPTDLYANHNRIFFDGTLRALDGMQGPIYVGTGCLFRRVTLYGFDPPRINVGGPCFPSLGGMFAKARYEKPGLEMTTKAAVAKGKHGFLPLPRKAYGKSEAFVDSIPRASHPSPFAAAAAVVADEATISEAVAVTTAAYEKKTGWGSNIGWVYGTVTEDVVTGYRMHIKGWRSRYCSIYPHAFIGTAPINLTERLYQVLRWSTGSLEIFFSRNNPLFGSTFLHPLQRVAYINITTYPFTAIFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSKQPAGDEKKDPYADLYVVRWTWLMVTPIIIILVNIVGSAVAFAKVVDGEWTHWLKVAGGVFFNFWVLFHLYPFAKGILGRHGKTPVVVLVWWAFTFVITAVLYINIPHIHGPGGKHAHGGAMGRHAHHHAGNNKFQEIYGWP

SEQ ID NO: 26 - protein, 939 aa, Brachypodium distachyon
MAPAVAGGSSRGAGCKCGFQVCVCSGSAAVASAGSSLEVERAMAVTPVEGQAAPVDGESWVGVELGPDGVETDESGAGVDDRPVFKTEKIKGVLLHPYRVLIFVRLIAFTLFVIWRISHKNPDTMWLWVTSICGEFWFGFSWLLDQLPKLNPINRIPDLAVLRQRFDRADGTSTLPGLDIFVTTADPIKEPILSTANSVLSILAADYPVDRNTCYISDDSGMLMTYEAMAESAKFATLWVPFCRKHGIEPRGPESYFELKSHPYMGRAHDEFVNDRRRVRKEYDDFKAKINSLETDIQQRNDLHNAAVPQNGDGIPRPTWMADGVQWQGTWVEPSANHRKGDHAGIVLVLIDHPSHDRLPGAPASADNALDFSGVDTRLPMLVYMSREKRPGHNHQKKAGAMNALTRASALLSNAPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPALGGLFAKTKYEKPSMEMTMARANQAVVPAMAKGKHGFLPLPKKTYGKSDKFVDTIPRASHPSPYAAEGIRVVDSGAETLAEAVKVTGSAFEQKTGWGSELGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLATLLIIAVLEVKWAGVTVFEWFRNGQFWMTASCSAYLAAVCQVLTKVIFRRDISFKLTSKLPAGDEKKDPYADLYVVRWTPLMITPIIIIFVNIIGSAVAFAKVLDGEWTHWLKVAGGVFFNFWVLFHLYPFAKGLLGKHGKTPVVVLVWWAFTFVITAVLYINIPHIHGGGGKHSVGHGMHHGKKFDGYYLWP

SEQ ID NO: 27 - protein, 944 aa, Avena magna
MAPAVAGGGRVRSNEAPAASAAAAATGNPCACGFQVCACTGTAAVASAASSVDMDIMATGRIGPLNDESWVGVELGEDGETDESGAAVDDRPVFRTEKIKAVLLYPYRVLIFVRLIAFTLFVIWRISHKNPDAMWLWVTSICGEFWFGFSWLLDQLPKLNPINRVPDLAVLRQRFDRPDGTSTLPGLDIFVTTADPFKEPILSTANSVLSILAADYPVDRNTCYVSDDSGMLLTYEALAEASKFATLWVPFCRKHGIEPRGPESYFELKSHPYMGRAQDEFVNDRRRVRKEYDEFKARINSLDHDIRQRNDGYNAANAHREGEPRPTWMADGTQWEGTWVDASENHRKGDHAGIVKVLLNHPSHSRQYGPPASANNPLDFSGVDVRVPMLVYVSREKRPGHNHQKKAGAMNALTRASALLSNAPFILNLDCDHYINNSQALRSGICFMLGRDSDTVAFVQFPQRFEGVDPTDLYANHNRIFFDGSLRALDGMQGPIYVGTGCLFRRITVYAFDPPRINVGGPCFPMLGGMFAKTKYQKPGLEMTMAKAKAAPVPAKGKHGFLPLPKKTYGKSDAFVDSIPLASHPSPYVAAYNTAEGIVTDEATMAEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRIAYINITTYPFTAIFLIFYTTVPALSFVTGHFIVQRPTTMFYVYLGIVLATLLIIAVLEVKWAGVTVFEWFRNGQFWMTASMSAYLQAVCQVLIKVIFQKDISFKLTSKLPAGDEKKDPYADLYVVRWTPLMIVPIIVIFVNIIGSAVAFAKVLDGEWTHWLKVAGGVFFNFWVLFHLYPFAKGILGKHGKTPVVVLVWWAFTFVITAVLYINIPHMHSPGGKHTKVAHGHHGQKFLGWP

SEQ ID NO: 28 - protein, 854 aa, Avena sativa
MAPAVAGGGRVRSNEAPAASAAAAATGNPCACGFQVCACTGTAAVASAASSVDMDIMATGRIGPLNDESWVGVELGEDGETDESGAAVDDRPVFRTEKIKAVLLYPYRVLIFVRLIAFTLFVIWRISHKNPDAMWLWVTSICGEFWFGFSWLLDQLPKLNPINRVPDLAVLRQRFDRPDGTSTLPGLDIFVTTADPFKEPILSTANSVLSILAADYPVDRNTCYVSDDSGMLLTYEALAEASKFATLWVPFCRKHGIEPRGPESYFELKSHPYMGRAQDEFVNDRRRVRKEYDEFKARINSLDHDIRQRNDGYNAANAHREGDPRPTWMADGTQWEGTWVDASENHRKGDHAGIVKVLLNHPSHSRQYAPASADNPLDFSGVDVRVPMLVYVSREKRPGHNHQKKAGAMNALTRASALLSNAPFILNLDCDHYINNSQALRSGICFMLGRDSDTVAFVQFPQRFEGVDPTDLYANHNRIFFDGSLRALDGMQGPIYVGTGCLFRRITVYAFDPPRINVGGPCFPMLGGMFAKTKYQKPGLEMTMAKAKAAPVPAKGKHGFLPLPKKTYGKSDAFVDSIPLASHPSPYVAAYNTAEGIVTDEATMAEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRIAYINITTYPFTAIFLIFYTTVPALSFVTGHFIVQRPTTMFYVYLGIVLATLLIIAVLEVKWAGVTVFEWFRNGQFWMTASMSAYLQAVCQVLIKVIFQKDISFKLTSKLPAGDEKKDPYADLYVVRWTPLMIVPIIVIFVNIIGSAVAFA

SEQ ID NO: 29 - protein, 801 aa, Triticum turgidum
MAPAVAGGGRVRSNEPAAAAASDKPCVCGFQVCACTGSAAVASAASSLDMDIVAMGQIGAANSVLSILAADYPVDRNTCYVSDDSGMLLTYEALAESSKFATLWVPFCRKHGIEPRGPESYFELKSHPYMGRAQDEFVNDRRRVRKEYDEFKARINSLEHDIKQRNDGYNAANAHREGEPRPTWMADGTQWEGTWVDASENHRRGDHAGIVRVLLNHPSHRRQTGPPASADNPLDFSGVDARLPMLVYVSREKRPGHDHQKKAGAMNALTRASALLSNSPFILNLDCDHYINNSQALRAGICFMVGRDSDTVAFVQFPQRFEGVDPTDLYANHNRIFFDGTLRALDGMQGPIYVGTGCLFRRITVYGFDPPRINVGGPCFPRLAGLFAKTKYEKPSLEMTMAKAKAAPVPAKGKHGFLPLPKKTYGKSDAFVDSIPRASHPSPYAAAAEGIVADEATIVEAVNVTAAAFEKKTGWGKEIGWVYDTVTEDVVTGYRMHIKGWRSRYCSIYPHAFIGTAPINLTERLFQVLRWSTGSLEIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSKLPSGDEKKDPYADLYVVRWTPLMITPIIIIFVNIIGSAVAFAKVLDGEWTHWLKVAGGVFFNFWVLFHLYPFAKGILGKHGKTPVVVLVWWAFTFVITAVLYINIPHMHSSGGKHTTVHGHHGKKFVDAGYYNWP

SEQ ID NO: 30 - protein, 133 aa, unidentified
EIFFSRHCPFWYGYGGGRLKWLQRLAYINTIVYPFTSLPLIAYCTIPAVCLLTGKFIIPTLSNLASMLFLGLFISIIVTAVLELRWSGVSIEDLWRNEQFWVIGGVSAHLFAVFQGFLKMLAGIDTNFTVTAK

SEQ ID NO: 31 - protein, 133 aa, Arabidopsis thaliana
EIFLSRHCPLWYGCSGGRLKLLQRLAYINTIVYPFTSLPLVAYCTLPAICLLTGKFIIPTLSNLASMLFLGLFISIILTSVLELRWSGVSIEDLWRNEQFWVIGGVSAHLFAVFQGFLKMLAGLDTNFTVTSK

SEQ ID NO: 32 - protein, 132 aa, Arabidopsis lyrata
EIFFSRHCPLWYAWGGKLKILERLAYINTIVYPFTSIPLLAYCTIPAVCLLTGKFIIPTINNFASIWFLALFLSIIATAILELRWSGVSINDLWRNEQFWVIGGVSAHLFAVFQGLLKVLFGVDTNFTVTSK

SEQ ID NO: 33 - protein, 132 aa, Arabidopsis thaliana
EIFLSRHCPIWYGYGGGLKWLERLSYINSVVYPWTSLPLIVYCSLPAICLLTGKFIVPEISNYASILFMALFSSIAITGILEMQWGKVGIDDWWRNEQFWVIGGVSAHLFALFQGLLKVLAGVDTNFTVTSK

SEQ ID NO: 34 - protein, 132 aa, Oryza sativa
EIFMSRHCPLWYAYGGRLKWLERFAYTNTIVYPFTSIPLLAYCTIPAVCLLTGKFIIPTLNNLASIWFIALFLSIIATGVLELRWSGVSIEDWWRNEQFWVIGGVSAHLFAVFQGLLKVLGGVDTNFTVTSK

SEQ ID NO: 35 - protein, 133 aa, Arabidopsis lyrata
EIFFSRHSPLWYGYKGGKLKWLERFAYANTTIYPFTSIPLLAYCILPAICLLTDKFIMPPISTFASLFFISLFMSIIVTGILELRWSGVSIEEWWRNEQFWVIGGISAHLFAVVQGLLKILAGIDTNFTVTSK

SEQ ID NO: 36 - protein, 132 aa, Arabidopsis thaliana
EILLSRHCPIWYGYHGRLRLLERIAYINTIVYPITSIPLIAYCILPAFCLITDRFIIPEISNYASIWFILLFISIAVTGILELRWSGVSIEDWWRNEQFWVIGGTSAHLFAVFQGLLKVLAGIDTNFTVTSK

SEQ ID NO: 37 - protein, 132 aa, Arabidopsis thaliana
EILLSRHCPIWYGYNGRLKLLERIAYINTIVYPITSIPLLAYCMLPAFCLITNTFIIPEISNLASLCFMLLFASIYASAILELKWSDVALEDWWRNEQFWVIGGTSAHLFAVFQGLLKVFAGIDTNFTVTSK

SEQ ID NO: 38 - protein, 130 aa, Hordeum vulgare
EIFFSKNNPLFGSTYLHPLQRVAYINITTYPFTAIFLIFYTTVPALSFVTGHFIVQRPTTMFYVYLGIVLSTLLVIAVLEVKWAGVTVFEWFRNGQFWMTASCSAYLAAVCQVLTKVIFRRDISFKLTSK

SEQ ID NO: 39 - protein, 130 aa, Oryza sativa
EIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVLQVVTKVVFRRDISFKLTSK

SEQ ID NO: 40 - protein, 130 aa, Eragrostis curvula
EIFFSKNNPLFGSTFLHPLQRVAYINITTYPFTAIFLIFYTTVPALSFVTGHFIVQRPTTMFYVYLLIVLGTLLILAVLEVKWAGVTVFEWFRNGQFWMTASCSAYLAAVLQVLFKVVFRRDISFKLTSK

SEQ ID NO: 41 - protein, 130 aa, Zea mays
EIFFSRNNPLFGSTFLHPLQRVAYINITTYPFTAIFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSK

SEQ ID NO: 42 - protein, 130 aa, Sorghum bicolor
EIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSK

SEQ ID NO: 43 - protein, 130 aa, Miscanthus lutarioriparius; identical to SEQ ID NO: 42

SEQ ID NO: 44 - protein, 130 aa, Setaria italica
EIFFSKNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVVVKVVFRRDISFKLTSK

SEQ ID NO: 45 - protein, 130 aa, Dichanthelium oligosanthes
EIFFSKNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVLVKVVFRRDISFKLTSK

SEQ ID NO: 46 - protein, 130 aa, Panicum hallii
EIFFSRNNPLFGSTFLHPLQRVAYINITTYPFTALFLIFYTTVPALSFVTGHFIVQRPTTMFYVYLAIVLGTLLILAVLEVKWAGVTVFEWFRNGQFWMTASCSAYLAAVCQVVVKVVFRRDISFKLTSK

SEQ ID NO: 47 - protein, 130 aa, Panicum virgatum; identical to SEQ ID NO: 41

SEQ ID NO: 48 - protein, 130 aa, Brachypodium distachyon
EIFFSKNNPLFGSTYLHPLQRVAYINITTYPFTAIFLIFYTTVPALSFVTGHFIVQRPTTMFYVYLGIVLATLLIIAVLEVKWAGVTVFEWFRNGQFWMTASCSAYLAAVCQVLTKVIFRRDISFKLTSK

SEQ ID NO: 49 - protein, 130 aa, Avena magna
EIFFSKNNPLFGSTYLHPLQRIAYINITTYPFTAIFLIFYTTVPALSFVTGHFIVQRPTTMFYVYLGIVLATLLIIAVLEVKWAGVTVFEWFRNGQFWMTASMSAYLQAVCQVLIKVIFQKDISFKLTSK

SEQ ID NO: 50 - protein, 130 aa, Avena sativa; identical to SEQ ID NO: 49

SEQ ID NO: 51 - protein, 130 aa, Triticum turgidum; identical to SEQ ID NO: 38

SEQ ID NO: 52 - protein, 130 aa, Prosopis alba
EIFFSRNNALLASPRMKLLQRIAYLNVGIYPFTSIFLIVYCFLPALSLFSGQFIVQTLNVTFLAYLLTITVTLCVLAILEIKWSGIELEEWWRNEQFWLIGGTSAHLAAVLQGLLKVIAGIEISFTLTSK

SEQ ID NO: 53 - protein, 130 aa, Glycine max
EIFFSRNNALLASPRMKILQRIAYLNVGIYPFTSIFLIVYCFLPALSLFSGQFIVQTLNVTFLSYLLGITVTLCMLAVLEIKWSGIELEEWWRNEQFWLIGGTSAHLAAVLQGLLKVVAGIEISFTLTSK

SEQ ID NO: 54 - protein, 130 aa, Pistacia vera
EIFFSRNNALLASPRMKFLQRIAYLNVGIYPFTSIFLIVYCFLPALSLFSGQFIVQTLNVTFLVYLLTITITLCLLAVLEIKWSGIELEEWWRNEQFWLIGGTSAHLAAVLQGLLKVVAGIEISFTLTSK

SEQ ID NO: 55 - protein, 130 aa, Gossypium hirsutum
EIFFSRNNALLASPRMKLLQRIAYLNVGIYPFTSIFLIVYCFLPALSLFSGQFIVQTLNVTFLTYLLIITVTLCLLAVLEIKWSGIELEEWWRNEQFWLIGGTSAHLAAVLQGLLKVIAGIEISFTLTSK

SEQ ID NO: 56 - protein, 130 aa, Hibiscus syriacus
EIFFSRNNALLASPRMKFLQRIAYLNVGIYPFTSIFLIVYCFLPALSLFSGQFIVQTLNVTFLTYLLIITVTLCLLAVLEIKWSGIELEEWWRNEQFWLIGGTSAHLAAVLQGLLKVIAGIEISFTLTSK

SEQ ID NO: 57 - protein, 130 aa, Herrania umbratica
EIFFSRNNAFLASPRMKLLQRIAYLNVGIYPFTSIFLIVYCFLPALSLFSGQFIVQTLNVTFLTYLLIITVTLCLLAVLEIKWSGIELEEWWRNEQFWLIGGTSAHLAAVLQGLLKVIAGIEISFTLTSK

SEQ ID NO: 58 - protein, 130 aa, Morella rubra
EIFFSRNNALLASPRMKFLQRIAYLNVGIYPFTSIFLIVYCFLPALSLFSGQFIVQTLSVTFLAYLLIISLTLCMLAILEIRWSGIELEEWWRNEQFWLIGGTSAHLAAVIQGLLKVLAGIEISFTLTSK

SEQ ID NO: 59 - protein, 130 aa, Quercus lobata
EIFFSRNNALLASPRMKLLQRIAYLNVGIYPFTSIFLIVYCFLPALSLFSGQFIVQTLNVTFLVYLLVITLTLCMLAILEIKWSGIELEEWWRNEQFWLIGGTSAHLAAVLQGLLKILAGIEISFTLTSK

SEQ ID NO: 60 - protein, 130 aa, Ensete ventricosum
EIFFSRNNALLASPRMKILQRIAYLNVGIYPFTSIFLIVYCFLPALSLFTGQFIVQTLNVTFLTYLLIITLTLCMLAVLEIKWSGIELEEWWRNEQFWLIGGTSAHLAAVLQGLLKVIAGIEISFTLTSK

SEQ ID NO: 61 - protein, 130 aa, Hordeum vulgare
EIFFSRNNALLASRRMKCLQRIAYLNVGIYPFTSIFLIVYCFLPALSLFSGQFIVKELDVTFLTYLLVITLTLCMLAVLEIKWSGINLEEWWRNEQFWLIGGTSAHLAAVLQGLLKVIAGIEISFTLTSK

SEQ ID NO: 62 - protein, 130 aa, Brachypodium distachyon
EIFFSRNNALLASRRMKFLQRIAYLNVGIYPFTSIFLIVYCFLPALSLFSGQFIVRTLDVTFLTYLLVITLTLCMLAVLEIKWSGINLEEWWRNEQFWLIGGTSAHLAAVLQGLLKVIAGIEISFTLTSK

SEQ ID NO: 63 - protein, 130 aa, Panicum virgatum
EIFFSRNNALLASRKMKFLQRIAYLNVGIYPFTSIFLIVYCFLPALSLFSGQFIVKQLNVTFLTYLLVITLTLCMLAVLEIKWSGISLEEWWRNEQFWLIGGTSAHLAAVLQGLLKVIAGIEISFTLTSK

SEQ ID NO: 64 - protein, 130 aa, Eragrostis curvula
EIFFSRNNALLASRRMKFLQRIAYLNVGIYPFTSIFLIVYCFLPALSLFSGQFIVQRLSVTFLTYLLVITLTLSMLAVLEIKWSGISLEEWWRNEQFWLIGGTSAHLAAVLQGLLKVIAGIEISFTLTSK

SEQ ID NO: 65 - DNA, 36 bp, synthetic construct
  source 1..36: mol_type = other DNA
ctatccacgctgctcgtcctcgccgtgctggaggtc

SEQ ID NO: 66 - DNA, 36 bp, synthetic construct
  source 1..36: mol_type = other DNA
gacctccagcacggcgaggacgagcagcgtggatag